SEQUENCE LISTING (56 sequences)

SEQ ID NO: 1 - DNA, 3372 bp, Homo sapiens
  CDS (155)..(3229)
gggccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccttcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaaagtgtttgtgcagaacatttgaggaagtacaataaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctccaaaaaaaaaaaaaaaaaaaa

SEQ ID NO: 2 - protein, 1025 aa, Homo sapiens [sequence omitted]

SEQ ID NO: 3 - DNA, 164 bp, Homo sapiens
aagatgatagtgatgagggtggtgatgatgagtattgtgatggtgatgaagatgaggatgatttaaagaaaccttgaaactggatgaaacagatagatttctcatgactttattttttgaaaacaataaaatgttgaaaaggctggctgaaaacccagaatatg

SEQ ID NO: 4 - DNA, 29 bp, Homo sapiens
tgatgagggtggtgatgatgagtattgtg

SEQ ID NO: 5 - DNA, 30 bp, Homo sapiens
gcagtgagttcaaacccatgacacagaatg

SEQ ID NO: 6 - DNA, 31 bp, Homo sapiens
cagcattctgaatagtcaagattgggaaatg

SEQ ID NO: 7 - DNA, 1284 bp, Homo sapiens
  misc_feature 1261: n = A,T,C or G
tgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttaccntcaaaaaaaaaaaaaaaaaaaaa

SEQ ID NO: 8 - DNA, 29 bp, Homo sapiens
cacaatactcatcatcaccaccctcatca

SEQ ID NO: 9 - DNA, 27 bp, Homo sapiens
gtagggccttattgtacttcctcaaat

SEQ ID NO: 10 - DNA, 1443 bp, Homo sapiens
  misc_feature 927: n = A,T,C or G
gaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccttcattatcattgatgaatgtcatcacaccaacaaagaagcagtgtataataacatcatgaggcattatttgatgcagaagttgaaaaacaatagactcaagaaagaaaacaaaccagtgattccccttcntcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaaagtgtttgtgcagaacatttgaggaagtacaataaggccctacaaattaatgacacaattcgaatgatagatgcgtatactcatcttgaaactttctataatgaagagaaagataagaagtttgcagtcatagaagatgatagtgatgagggtggtgatgatgagtattg

SEQ ID NO: 11 - DNA, 21 bp, Homo sapiens
ctccaacaccaggtgaagctg

SEQ ID NO: 12 - DNA, 21 bp, Homo sapiens
cagatgaagagaatgtggcag

SEQ ID NO: 13 - DNA, 24 bp, Homo sapiens
ggaagtacaatgagggcctacaaa

SEQ ID NO: 14 - DNA, 24 bp, Homo sapiens
tcctcagtcctagtatattgctcc

SEQ ID NO: 15 - DNA, 39 bp, Homo sapiens
ctaagcagctgacacttccttctgccaaacttgtgtctg

SEQ ID NO: 16 - DNA, 27 bp, Homo sapiens
gggccctgtggacaacctcgtcattgt

SEQ ID NO: 17 - DNA, 33 bp, Homo sapiens
ccagagtggctgtttacattgccaaggatcact

SEQ ID NO: 18 - DNA, 30 bp, Homo sapiens
gcatctgcaatggcaaacttcttgcatggc

SEQ ID NO: 19 - DNA, 24 bp, Homo sapiens
gccatcaatgaccccttcattgac

SEQ ID NO: 20 - DNA, 24 bp, Homo sapiens
tgacgaacatgggggcatcagcag

SEQ ID NO: 21 - DNA, 30 bp, Homo sapiens
tgagaggatccgatgtcgaatgggtattcc

SEQ ID NO: 22 - DNA, 30 bp, Homo sapiens
aatgtcgacctaatcctcatcactaaataa

SEQ ID NO: 23 - DNA, 36 bp, Homo sapiens
tgagagctcgagatgtcgaatgggtattccacagac

SEQ ID NO: 24 - DNA, 36 bp, Homo sapiens
tgtttatttagtgatgaggatcgggatccgattgaa

SEQ ID NO: 25 - DNA, 33 bp, Homo sapiens
ttcaatctcgagatcctcatcactaaataaaga

SEQ ID NO: 26 - DNA, 30 bp, Homo sapiens
cgtgctgattcctcagtcctagtatattgc

SEQ ID NO: 27 - DNA, 13 bp, Artificial sequence
  Description of the artificial sequence Primer
tatcgactccaag

SEQ ID NO: 28 - DNA, 13 bp, Artificial sequence
  Description of the artificial sequence Primer
ttagctagcatgg

SEQ ID NO: 29 - DNA, 13 bp, Artificial sequence
  Description of the artificial sequence Primer
tgctaagactagc

SEQ ID NO: 30 - DNA, 13 bp, Artificial sequence
  Description of the artificial sequence Primer
ttgcagtgtgtga

SEQ ID NO: 31 - DNA, 13 bp, Artificial sequence
  Description of the artificial sequence Primer
tgtgaccattgca

SEQ ID NO: 32 - DNA, 13 bp, Artificial sequence
  Description of the artificial sequence Primer
tgtctgctaggta

SEQ ID NO: 33 - DNA, 13 bp, Artificial sequence
  Description of the artificial sequence Primer
tgcatggtagtct

SEQ ID NO: 34 - DNA, 13 bp, Artificial sequence
  Description of the artificial sequence Primer
tgtgttgcaccat

SEQ ID NO: 35 - DNA, 13 bp, Artificial sequence
  Description of the artificial sequence Primer
tagacgctagtgt

SEQ ID NO: 36 - DNA, 13 bp, Artificial sequence
  Description of the artificial sequence Primer
ttagctagcagac

SEQ ID NO: 37 - DNA, 13 bp, Artificial sequence
  Description of the artificial sequence Primer
tcatgatgctacc

SEQ ID NO: 38 - DNA, 13 bp, Artificial sequence
  Description of the artificial sequence Primer
tactccatgactc

SEQ ID NO: 39 - DNA, 13 bp, Artificial sequence
  Description of the artificial sequence Primer
tattacaacgagg

SEQ ID NO: 40 - DNA, 13 bp, Artificial sequence
  Description of the artificial sequence Primer
tattggattggtc

SEQ ID NO: 41 - DNA, 13 bp, Artificial sequence
  Description of the artificial sequence Primer
tatctttctaccc

SEQ ID NO: 42 - DNA, 13 bp, Artificial sequence
  Description of the artificial sequence Primer
tatttttggctcc

SEQ ID NO: 43 - DNA, 13 bp, Artificial sequence
  Description of the artificial sequence Primer
ttatctatacagg

SEQ ID NO: 44 - DNA, 13 bp, Artificial sequence
  Description of the artificial sequence Primer
ttatggtaaaggg

SEQ ID NO: 45 - DNA, 13 bp, Artificial sequence
  Description of the artificial sequence Primer
ttatcggtcatag

SEQ ID NO: 46 - DNA, 13 bp, Artificial sequence
  Description of the artificial sequence Primer
ttaggtactaagg

SEQ ID NO: 47 - DNA, 24 bp, Homo sapiens
gaaagagaggtcgcagaggcctgt

SEQ ID NO: 48 - DNA, 24 bp, Homo sapiens
tgataaggctgaggaagggaaatg

SEQ ID NO: 49 - DNA, 21 bp, Homo sapiens
ctagacccctggaagcatcca

SEQ ID NO: 50 - DNA, 21 bp, Homo sapiens
tcgggcctgtcgggtcccctc

SEQ ID NO: 51 - DNA, 20 bp, Homo sapiens
gggtcagaaggattcctatg

SEQ ID NO: 52 - DNA, 20 bp, Homo sapiens
ggtctcaaacatgatctggg

SEQ ID NO: 53 - DNA, 27 bp, Human immunodeficient virus type 1
  misc_feature 3, 6, 12, 18: n = inosine
gcnttnagcccngaagtnatacccatg

SEQ ID NO: 54 - DNA, 28 bp, Human immunodeficient virus type 1
  misc_feature 4, 15: n = inosine
catnctatttgttcntgaagggtactag

SEQ ID NO: 55 - DNA, 28 bp, Human immunodeficient virus type 1
  misc_feature 11, 16, 20: n = inosine
ggcttgctgangngcncacngcaagagg

SEQ ID NO: 56 - DNA, 28 bp, Human immunodeficient virus type 1
  misc_feature 6: n = inosine
agagtngtggttgnttcnttccacacag

(19) United States
(12) Patent Application Publication
(10) Pub. No.: US 2004/0086500 A1
(43) Pub. Date: May 6, 2004

(54) Rh116 polypeptides and its fragments and polynucleotides encoding said polypeptides and therapeutic uses

(76) Inventors: Georges Bahr, Lille (FR); Cecile Cocude, Annoeullin (FR); Andre Capron, Phalempin (FR)

Correspondence Address: BURNS DOANE SWECKER & MATHIS L L P, POST OFFICE BOX 1404, ALEXANDRIA, VA, 22313-1404, US

(21) Appl. No.: 10/275,822

(22) PCT Filed: May 11, 2001
(86) PCT No.: PCT/FR01/01441

(30) Foreign Application Priority Data
May 11, 2000 (FR) 00/06030

(51) Int. Cl. A61K 48/00; C12Q 1/68; C07H 21/04; C12N 9/12

(52) U.S. Cl. 424/94.5; 435/6; 435/69.1; 435/194; 435/320.1; 435/325; 530/388.26; 514/44; 536/23.2

(57) ABSTRACT

The invention concerns a novel 116 kDa polypeptide exhibiting homologies of sequences with RNA helicases (DEXH box) called RH116 and its fragments, the cloning of cDNA and the polynucleotides encoding said polypeptides, cloning and/or expression vectors including said polynucleotides, cells transformed by said vectors and specific antibodies directed against said polypeptides. The invention also concerns methods for detecting and/or assaying said polypeptides and polynucleotides, corresponding diagnosis kits, and a method for screening ligands, as well as compounds for use as medicine for preventing and/or therapeutic treatment.

The present invention relates to a novel 116 kDa polypeptide exhibiting sequence homologies with RNA helicases (DEXH box), named RH116, and to its fragments, to the cloning of the cDNA and the polynucleotides encoding said polypeptides, to cloning and/or expression vectors including said polynucleotides, to cells transformed with said vectors and to specific antibodies against said polypeptides. The invention also relates to methods for detecting and/or assaying said polypeptides and polynucleotides, to the corresponding diagnostic kits, and to a method for screening ligands and also compounds which can be used as a medicinal product for prevention and/or therapeutic treatment.

Muramyl peptides are, among synthetic immunomodulators, those which have shown a large number of immunopharmacological effects on cells of the monocyte/macrophage line, potentiating their nonspecific resistance to infection, increasing the tumoricidal activity of macrophages, and also acting as vaccine adjuvants. Murabutide (MB), an analog of muramyl dipeptide (MDP), has been selected for its particularly promising biological profile and its good tolerance in animals and in humans. Specifically, unlike MDP and many other analogs, it has been demonstrated that MB is not pyrogenic, does not induce inflammatory reactions and has not shown severe toxicity in clinical studies in healthy volunteers and patients suffering from cancer.

By virtue of its biological capacities, MB is an antiviral agent which is promising in the AIDS (acquired immunodeficiency syndrome) field. Specifically, MB inhibits human immunodeficiency virus (HIV) replication in macrophages and dendritic cells, but also in peripheral blood mononuclear cells (PBMCs) of infected patients. Thus, given these biological characteristics, the immunomodulator MB was the subject of a granted French patent No. FR 2 724 845 entitled “Compositions of muramyl peptides capable of inhibiting up to 100% the replication of an acquired immunodeficiency virus such as HIV”. In addition, phase I and phase IIa clinical trials carried out to the end on HIV+ patients have demonstrated good clinical tolerance of MB.

The inventors have demonstrated that MB exerts a strong inhibition of viral replication in the PBMCs of CD8 lymphocyte-depleted patients, activated with phytohemagglutinin (PHA) and cultured with interleukin 2 (IL-2). Specifically, MB inhibits by 70 to 100% the level of HIV viral protein p24 in the culture supernatants. This effect correlates with the level of expression of viral messenger RNAs (nonspliced and single-spliced). In addition, analysis of the profile of secreted cytokines and chemokines has demonstrated that MB induces reproduction of chemokines known to be inhibitors of HIV replication. However, this induction does not appear to correlate completely with the inhibitory effect of MB. The inhibition of HIV replication by MB does not only involve induction of β-chemokine production, since MB is also involved at the level of the proviral DNA and of viral transcription. The lack of toxicity of MB in these same cell cultures has been verified by the inventors, who noted that not only does the number of live cells remain unchanged at the start of culturing, but it also appears to increase at the end of culturing.

The results obtained by the inventors therefore suggest that MB induces the production of cytokines or other factors not identified to date, which have a suppressor activity on HIV replication.

In order to identify these new factors involved in regulating viral replication, the inventors have used the “Differential Display-RT-PCR” (DD-RT-PCR) methodology, which is based on 2 essential steps; a first step of reverse transcription (RT) of the total cellular RNA in order to obtain complementary DNAs of all the RNAs which have a poly A tail, and then a second step of amplification by polymerase chain reaction (PCR) using the cDNAs, which serve as matrix, and various pairs of primers in the presence of a radiolabeled nucleotide. The PCR products are then separated on a gel by electrophoresis. The differentially amplified fragments are cut out from the gel, reamplified and then cloned and sequenced.

DD-RT-PCR, carried out using PBMCs from an HIV+ patient, has made it possible for the inventors to select more than 130 cDNA fragments differentially expressed after treatment with MB. These fragments were subcloned into vector pCR2.1 (Invitrogen), then sequenced by automatic sequencing (ABI Prism 377, Perkin-Elmer). The sequences were analyzed for homology searches using the databanks and the Basic Local Alignment Search Tool (Blast 2) server of the NCBI.

The inventors have identified a novel polypeptide exhibiting sequence homologies with RNA helicases.

RNA helicases (for review see Critical Rev. in Biochemistry and Molecular Biology (1998) 33 (4): 259-296) represent a large family of proteins present in all types of biological system in which RNA plays a central role. They are distributed ubiquitously in a wide range of organisms and are involved in the mitochondrial and nuclear splicing process, RNA editing, rRNA processing, translation initiation, export of nuclear mRNAs and degradation of mRNAs.

RNA helicases constitute factors which are essential to cell differentiation and development, and some of them play a role in single-stranded RNA viral genome transcription and replication.

RNA helicases belong to the large group of enzymes capable of hydrolyzing nucleotide 5′-tri-phosphates.

A sequence comparison study of DNA-dependent ATPases has led to a new classification of NTPases according to the ATPase A motif. Thus, DNA helicases are characterized by an A motif, also called a “Walker” motif (G-X-X-X-X-G-K-T), and belong to the superfamily I, whereas RNA helicases exhibit variations in this domain (A-X-X-G-X-G-K-T) and form the close superfamily II (Gorbalenya et al., 1988).

Comparisons of the conserved sequences have shown close links between the various RNA helicases, suggesting that these proteins derive from a common ancestor.

In fact, alignment of the amino acid sequence of various members of the RNA helicase superfamily II has made it possible to demonstrate a common central region which is characterized by the presence of eight highly conserved domains. At the current time, the biochemical function of four of the eight conserved domains (domains I, II, VI and VIII) have been elucidated. Given the strong sequence homology in the fifth of the eight structural elements, called DEAD box motif (D-E-A-D: Asp-Glu-Ala-Asp), the RNA helicases belonging to superfamily II are also called “DEAD box” proteins (Linder et al., 1989). The existence of divergent DEAD motifs has made it possible to subdivide the RNA helicase superfamily II into subgroups. To date, three subgroups have been identified. The first subgroup is made up of the conventional DEAD box proteins, the other two subgroups are called DEAH and DEXH because of their divergent ATPase B motif.

On either side of the conserved central sequence, the amino- and carboxy-terminal ends of RNA helicases are characterized by sequences of varying length and content. It is suggested that these divergent regions are responsible for the individual protein functions, whereas the highly conserved domains are involved in the RNA helicase activity.

Domain I (A/G-X-X-G-X-G-K-T: Ala/Gly-X-X-Gly-X-Gly-lys-Thr) is described as being the A motif of ATPases (Walker et al., 1982).

Domain V, or DEAD box (L-D-E-A-D-X-X-Leu: Leu-Asp-Glu-Ala-Asp-X-X-leu) represents a specific form of the ATPase B motif (Walker et al., 1982) which appears to be involved in ATP hydrolysis (Pause and Sonenbery, 1992).

Domain VI or SAT motif (Ser-Ala-Thr) is located in the proximity of the DEAD box and appears to be specific for RNA helicases.

Domain VIII is characterized by the YIHRIGRXXR box (Tyr-Ile-His-Arg-Ile-Gly-Arg-X-X-Arg), which represents a motif which is, like SAT, specific for RNA helicases. In vitro experiments with the translation initiation factor eIF-4A indicate that this domain is critical for RNA binding.

A subject of the present invention is therefore an isolated polypeptide, named “RH116” (for RNA helicase of 116 kDa), of amino acid sequence SEQ ID No. 2. This sequence comprises conserved consensus domains which are easily identifiable by those skilled in the art and which make it possible to classify the RH116 polypeptide of the invention in the DEAH or DEXH subgroup (for review see Luking et al., (1998)). Among these conserved consensus domains, mention should be made of:

The GSGKT sequence which corresponds to amino acids 332 to 336 of the sequence SEQ ID No. 2, and which constitutes conserved domain I (G-GKT) of the RNA helicase superfamily.

The DECH sequence which corresponds to amino acids 443 to 446 of the sequence SEQ ID No. 2, and which constitutes conserved domain V (DE-H) of the superfamily of RNA helicases of the DEXH subgroup.

These two conserved consensus domains are involved in the ATPase function.

The TAS sequence which corresponds to amino acids 488 to 490 of the sequence SEQ ID No. 2, and which constitutes conserved domain VI (-A-) of the superfamily of RNA helicases of the DEAH subgroup.

The RGRAR sequence which corresponds to amino acids 820 to 824 of the sequence SEQ ID No. 2, and which constitutes conserved domain VIII of the superfamily of RNA helicases of the DEAH subgroup.

These two conserved domains (domains VI and VIII) are more specific for RNA helicases and are responsible for the binding and for the unfolding of the target RNA.

The isolated polypeptide is characterized in that it comprises a polypeptide chosen from:

a) a polypeptide of sequence SEQ ID No. 2;

b) a variant polypeptide of the polypeptide of amino acid sequences defined in a);

c) a polypeptide homologous to the polypeptide defined in a) or b) and comprising at least 80% identity, preferably 85%, 87%, 90%, 95%, 97%, 98%, 99% identity, with said polypeptide of a);

d) a fragment of at least 15 consecutive amino acids, preferably 17, 20, 23, 25, 30, 40, 50, 100, 250 consecutive amino acids, of a polypeptide defined in a), b) or c);

e) a biologically active fragment of a polypeptide defined in a), b) or c).

In the present description, the term “polypeptide” will be used to denote a protein or a peptide equally.

The term “variant polypeptide” will be intended to mean all of the mutated polypeptides which can exist naturally, in particular in humans, and which correspond in particular to truncations, substitutions, deletions and/or additions of amino acid residues.

The term “homologous polypeptide” will be intended to denote the polypeptides having, compared with the natural RH116 polypeptide, certain modifications, such as in particular a deletion, addition or substitution of at least one amino acid, a truncation, an extension and/or a chimeric fusion. Among the homologous polypeptides, preference is given to those the amino acid sequence of which exhibits at least 80% identity, preferably at least 85%, 87%, 90%, 93%, 95%, 97%, 98%, 99% identity, with the amino acid sequences of the polypeptides according to the invention. In the case of a substitution, one or more consecutive or nonconsecutive amino acids can be replaced with “equivalent” amino acids. The expression “equivalent” amino acid is herein intended to denote any amino acid capable of substituting for one of the amino acids of the basic structure without, however, modifying the essential functional properties or characteristics, for instance their biological activities, of the corresponding polypeptides, such as the in vivo induction of antibodies capable of recognizing the polypeptide the amino acid sequence of which is included in the amino acid sequence SEQ ID No. 2, or one of its fragments. These equivalent amino acids can be determined either based on their structural homology with the amino acids for which they substitute, or on the results of assays for cross biological activity which the various polypeptides are liable to produce. By way of example, mention will be made of the possibilities of substitutions which can be made without a profound modification of the biological activities of the corresponding modified polypeptides resulting therefrom; replacement, for example, of leucine with valine or isoleucine, of aspartic acid with glutamic acid, of glutamine with asparagine, of arginine with lysine, etc., it naturally being possible to envision the reverse substitutions under the same conditions.

The expression “biologically active fragment” will be intended to denote in particular a fragment of amino acid sequence of a polypeptide according to the invention having at least one of the functional properties or characteristics of the polypeptide according to the invention, in particular in that it comprises RNA helicase activity. The variant polypeptide, the homologous polypeptide or the polypeptide fragment according to the invention has at least 10%, preferably 20%, 30%, 40%, 50%, 60%, 70%, 80%, 90%, 95%, of the RNA helicase activity. Various protocols known to those skilled in the art have been described for measuring the RNA helicase activity of the polypeptides according to the invention; mention should be made of the articles by Lain et al. (1990) and Lee and Hurwitz (1993). The examples below propose biological functions for the RH116 protein according to the peptide domains of this protein, and thus make it possible for a person skilled in the art to identify the biologically active fragments.

The term “polypeptide fragment” is intended to denote a polypeptide comprising a minimum of 15 consecutive amino acids, preferably 17, 20, 23, 25, 30, 40, 50, 100, 250 consecutive amino acids. The polypeptide fragments according to the invention obtained by cleaving said polypeptide with a proteolytic enzyme or with a chemical reagent, or else by placing said polypeptide in a very acidic environment, are also part of the invention.

Preferably, a polypeptide according to the invention is a polypeptide consisting of the sequence SEQ ID No. 2 or of a sequence having at least 80% identity, preferably at least 85%, 90%, 95%, 98% and 99% identity, with SEQ ID No. 2 after optimal alignment. The expression “polypeptide the amino acid sequence of which exhibits a percentage identity of at least 80%, preferably of at least 85%, 90%, 95%, 98% and 99%, after optimal alignment, with a reference sequence” is intended to denote the polypeptides having certain modifications compared with the reference polypeptide, such as in particular one or more deletions or truncations, an extension, a chimeric fusion and/or one or more substitutions.

Among the polypeptides the amino acid sequence of which exhibits a percentage identify of at least 80%, preferably of at least 85%, 90%, 95%, 98% and 99%, after optimal alignment, with the sequences SEQ ID No. 2 or with one of their fragments according to the invention, preference is given to the variant polypeptides encoded by the variant peptide sequences as defined above, in particular the polypeptides the amino acid sequence of which has at least one mutation corresponding in particular to a truncation, deletion, substitution and/or addition of at least one amino acid residue compared with the sequences SEQ ID No. 2 or with one of their fragments, more preferably the variant polypeptides having a mutation associated with a pathology.

The polypeptide according to the invention is characterized in that it comprises at least one conserved domain belonging to the RNA helicase superfamily, these domains preferably being chosen from the G-GKT sequences corresponding to domain I, the DEAD, DE-D and DEAH sequences corresponding to domain V, the SAT and -A- sequences corresponding to domain VI, and the YIHRIGRR, HRIGR—R, —R-GR—R and -GR sequences corresponding to domain VIII.

The invention also relates to a purified or isolated polynucleotide, characterized in that it encodes a polypeptide of sequence SEQ ID No. 2 as defined above. Preferably, the polynucleotide according to the invention has the sequence SEQ ID No. 1.

The purified or isolated polynucleotide according to the invention is characterized in that it comprises a polynucleotide chosen from:

a) SEQ ID No. 1;

b) the sequence of a fragment of at least 15 consecutive nucleotides, preferably of at least 18, 21, 24, 27, 30, 35, 40, 50, 75, 100 consecutive nucleotides, of the sequence SEQ ID No. 1, with the exception of the nucleic acid sequences identified under the accession No. AC 007750 and No. AC0108176 in the GenBank databank, and also with the exception of the genomic sequence of 95417 bp, and also with the exception of the registered nucleic acid sequences identified under the accession Nos. AW589567, AW152541 and AW189548 in the EMBL databank;

c) a nucleic acid sequence exhibiting a percentage identity of at least 80%, preferably of at least 85%, 90%, 95%, 98% and 99%, after optimal alignment, with a sequence defined in a) or b);

d) the complementary sequence or the RNA sequence corresponding to a sequence as defined in a), b) or c).

The terms “nucleic acid”, “nucleic acid sequence”, “polynucleotide”, “oligonucleotide”, “polynucleotide sequence” and “nucleotide sequence”, all terms which will be used equally in the present description, are intended to denote a precise series of nucleotides, which may or may not be modified, making it possible to define a fragment or a region of a nucleic acid, which may or may not comprise unnatural nucleotides, and which may correspond equally to a double-stranded DNA, a single-stranded DNA and transcription products of said DNAs, and/or an RNA fragment.

It should be understood that the present invention does not relate to the nucleotide sequences in their natural chromosomal environment, that is to say in the natural state. They are sequences which have been isolated and/or purified, that is to say they have been taken directly or indirectly, for example by copying, their environment having been at least partially modified. Thus, nucleic acids obtained by chemical synthesis are also intended to be denoted.

The expression “polynucleotide of complementary sequence” is intended to denote any DNA the nucleotides of which are complementary to those of SEQ ID No. 1, or of a portion of SEQ ID No. 1, and the orientation of which is reversed.

For the purpose of the present invention, the term “percentage identity” between two nucleic acid or amino acid sequences is intended to denote a percentage of nucleotides or of amino acid residues which are identical between the two sequences to be compared, obtained after the best alignment, this percentage being purely statistical and the differences between the two sequences being distributed randomly and over their entire length. The term “best alignment” or “optimal alignment” is intended to denote the alignment for which the percentage identity determined as below is highest. The sequence comparisons between two nucleic acid or amino acid sequences are conventionally carried out by comparing these sequences after having aligned them optimally, said comparison being carried out by segment or by “window of comparison” so as to identify and compare the local regions of sequence similarity. The optimal alignment of the sequences for the comparison may be carried out, besides manually, by means of the local homology algorithm of Smith and Waterman (1981), by means of the local homology algorithm of Neddleman and Wunsch (1970), by means of the similarity search method of Pearson and Lipman (1988), by means of computer programs using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimum alignment, the BLAST program is preferably used, with the BLOSUM 62 matrix. The PAM or PAM250 matrices may also be used.

The percentage identify between two nucleic acid or amino acid sequences is determined by comparing these two sequences aligned optimally, the nucleic acid or amino acid sequence to be compared possibly comprising additions or deletions with respect to the reference sequence for optimal alignment between these two sequences. The percentage identity is calculated by determining the number of identical positions for which the nucleotide or the amino acid residue is identical between the two sequences, dividing this number of identical positions by the total number of positions compared and multiplying the result obtained by 100 so as to obtain the percentage identity between these two sequences.

The expression “nucleic acid sequences exhibiting a percentage identity of at least 80%, preferably of at least 85%, 90%, 95%, 98% and 99%, after optimal alignment, with a reference sequence” is intended to denote the nucleic acid sequences which, compared to the reference nucleic acid sequence, have certain modifications, such as in particular a deletion, a truncation, an extension, a chimeric fusion and/or a substitution, in particular of the point type, and the nucleic acid sequence of which exhibits at least 80%, preferably at least 85%, 90%, 95%, 98% and 99%, identity, after optimal alignment, with the reference nucleic acid sequence. They are preferably sequences whose complementary sequences are capable of hybridizing specifically with the sequences SEQ ID No. 1 of the invention. Preferably, the specific or high stringency hybridization conditions will be such that they ensure at least 80%, preferably at least 85%, 90%, 95%, 98% and 99%, identity, after optimal alignment, between one of the two sequences and the sequence complementary to the other.

Hybridization under high stringency conditions means that the conditions of temperature and of ionic strength are chosen such that they allow the hybridization between two complementary DNA fragments to be maintained. By way of illustration, high stringency conditions for the hybridization step for the purposes of defining the polynucleotide fragments described above are advantageously as follows:

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10× Denhardt's, 5% of dextran sulfate and 1% of salmon sperm DNA; (2) hybridization per se for 20 hours at a temperature which depends on the length of the probe (i.e.: 42° C. for a probe >100 nucleotides in length), followed by 2 washes of 20 minutes at 20° C. in 2×SSC+2% SDS and 1 wash of 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length may be adjusted by those skilled in the art for longer or shorter oligonucleotides, according to the teaching of Sambrook et al., 1989.

Among the nucleic acid sequences exhibiting a percentage identity of at least 80%, preferably of at least 85%, 90%, 95%, 98% and 99%, after optimal alignment, with the sequence according to the invention, preference is also given to the nucleic acid sequences which are variants of SEQ ID No. 1, or of their fragments, that is to say all the nucleic acid sequences corresponding to allelic variants, that is to say individual variations of the sequences SEQ ID No. 1. These natural mutated sequences correspond to polymorphisms present in mammals, in particular in humans, and especially to polymorphisms which may lead to the occurrence of a pathology.

The expression “variant nucleic acid sequence” is also intended to denote any RNA or cDNA resulting from a mutation and/or variation of a splice site of the genomic nucleic acid sequence the cDNA of which has the sequence SEQ ID No. 1.

More particularly, the invention relates to a purified or isolated nucleic acid according to the present invention, characterized in that it comprises or consists of one of the sequences SEQ ID No. 1, of the sequences complementary thereto, or of the RNA sequences corresponding to SEQ ID No. 1. The primers or probes, characterized in that they comprise a nucleic acid sequence according to the invention, are also part of the invention. Thus, the present invention for detecting, identifying, assaying or amplifying a nucleic acid sequence also relates to the primers or the probes according to the invention which may make it possible in particular to demonstrate or to distinguish the variant nucleic acid sequences, or to identify the genomic sequence of the genes the cDNA of which is represented by SEQ ID No. 1, in particular using an amplification method such as the PCR method or a related method. According to the invention, the polynucleotides which can be used as a probe or as a primer in methods for detecting, identifying, assaying or amplifying a nucleic acid sequence are a minimum of 15 bases, preferably at least 18, 20, 25, 30, 40, 50 bases, in length.

The polynucleotides according to the invention may thus be used as a primer and/or probe in methods using in particular the PCR (polymerase chain reaction) technique (Rolfs et al., 1991). This technique requires choosing pairs of oligonucleotide primers bordering the fragment which must be amplified. Reference may, for example, be made to the technique described in U.S. Pat. No. 4,683,202. The amplified fragments can be identified, for example after agarose or polyacrylamide gel electrophoresis, or after a chromatographic technique such as gel filtration or ion exchange chromatography, and then sequenced. The specificity of the amplification can be controlled using, as primers, the nucleotide sequences of polynucleotides of the invention and, as matrices, plasmids containing these sequences or else the derived amplification products. The amplified nucleotide fragments may be used as reagents in hybridization reactions in order to demonstrate the presence, in a biological sample, of a target nucleic acid of sequence complementary to that of said amplified nucleotide fragments. The invention is also directed toward the nucleic acids which can be obtained by amplification using primers according to the invention.

Other techniques for amplifying the target nucleic acid may advantageously be employed as an alternative to PCR (PCR-like) using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, they involve amplifying the DNA with a polymerase; when the sample of origin is an RNA, a reverse transcription should be carried out beforehand. A large number of methods currently exist for this amplification, such as, for example, the SDA (strand displacement amplification) technique (Walker et al., 1992), the TAS (transcription-based amplification system) technique described by Kwoh et al. (1989), the 3SR (self-sustained sequence replication) technique described by Guatelli et al. (1990), the NASBA (nucleic acid sequence based amplification) technique described by Kievitis et al. (1991), the TMA (transcription mediated amplification) technique, the LCR (ligase chain reaction) technique described by Landegren et al. (1988), the RCR (repair chain reaction) technique described by Segev (1992), the CPR (cycling probe reaction) technique described by Duck et al. (1990), and the Q-beta-replicase amplification technique described by Miele et al. (1983). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA obtained in the biological sample. The cDNA obtained will then serve as a target for the primers or the probes used in the amplification or detection method according to the invention.

The probe hybridization technique may be carried out in various ways (Matthews et al., 1988). The most general method consists in immobilizing the nucleic acid extracted from the cells of various tissues or from cells in culture, on a support (such as nitrocellulose, nylon or polystyrene), so as to produce, for example, DNA chips, and then in incubating the immobilized target nucleic acid with the probe, under well-defined conditions. After hybridization, the excess probe is removed and the hybrid molecules formed are detected using the appropriate method (measuring the radioactivity, the fluorescence or the enzymatic activity linked to the probe).

According to another embodiment of the nucleic acid probes according to the invention, the latter may be used as capture probes. In this case, a probe, termed “capture probe”, is immobilized on a support and is used to capture, by specific hybridization, the target nucleic acid obtained from the biological sample to be tested, and the target nucleic acid is then detected using a second probe, termed “detection probe”, labeled with a readily detectable element.

Among the advantageous nucleic acid fragments, mention should, moreover, be made in particular of antisense oligonucleotides, i.e. oligonucleotides the structure of which ensures, by hybridization with the target sequence, inhibition of expression of the corresponding product. Mention should also be made of sense oligonucleotides which, by interacting with proteins involved in regulating the expression of the corresponding product, will induce either inhibition or activation of this expression. The oligonucleotides according to the invention are a minimum of 9 bases in length, preferably at least 10, 12, 15, 17, 20, 25, 30, 40, 50 bases in length.

The probes, primers and oligonucleotides according to the invention may be labeled directly or indirectly with a radioactive or nonradioactive compound, by methods well known to those skilled in the art, in order to obtain a detectable and/or quantifiable signal. The polynucleotide sequences according to the invention which are unlabeled may be used directly as a probe or primer.

The sequences are generally labeled so as to obtain sequences which can be used for many applications. The primers or probes according to the invention are labeled with radioactive elements or with nonradioactive molecules. Among the radioactive isotopes used, mention may be made of ³²P, ³³P, ³⁵S, ³H or ¹²⁵I. The nonradioactive entities are selected from ligands such as biotin, avidin, streptavidin or dioxygenin, haptens, dyes and luminescent agents, such as radioluminescent, chemiluminescent, bioluminescent, fluorescent or phosphorescent agents.

The present invention also relates to the cloning and/or expression vectors comprising a nucleic acid or encoding a polypeptide according to the invention. Such a vector may also contain the elements required for the expression and, optionally, the secretion of the polypeptide in a host cell. Such a host cell is also a subject of the invention.

According to another aspect, the invention relates to an antisense expression vector. Such an expression vector contains a polynucleotide sequence according to the invention, inserted in reverse orientation into the expression vector. Thus, those skilled in the art readily recognize that an mRNA corresponding to the DNA in the antisense vector hybridizes with an mRNA corresponding to DNA in the sense vector. An antisense expression vector is a vector which expresses an antisense RNA of interest in a suitable host cell, either constitutively or after induction. The term “antisense” refers to any composition containing a specific nucleic acid sequence. The antisense molecules can be produced by methods such as synthesis or transcription. When such molecules are introduced into the cell, the complementary nucleotides combine with the natural sequences produced by the cell to form duplexes and thus block either the transcription or the translation of the polypeptide according to the invention. It is also within the scope of the invention to produce antisense molecules capable of pairing with the RNA molecule which is the substrate for the RH116 RNA helicase, in order to block the biological activity thereof.

The novel compounds identified which are capable of decreasing or destroying the level of expression and/or the RNA helicase activity of the polypeptide according to the invention constitute antagonists of the polypeptide according to the invention. The term “antagonist” refers to a molecule which, when it binds to the polypeptide according to the invention, decreases the amount or the duration of the effects of the biological or immunological activity of the RH116 polypeptide. The antagonists include proteins, nucleic acids, carbohydrates and all molecules capable of decreasing the effects of RH116.

Said vectors preferably comprise a promoter, translation initiation and termination signals, and also regions suitable for regulating transcription. It must be possible for them to be maintained stably in the cell and they may optionally possess particular signals specifying secretion of the translated protein. According to a particular embodiment of the invention, the promoter may be the promoter naturally present upstream of the gene encoding the human RH116 polypeptide of the invention.

The various control signals are chosen as a function of the cellular host used. To this effect, the nucleic acid sequences according to the invention may be inserted into vectors which replicate autonomously in the chosen host, or vectors which integrate in the chosen host.

Among the systems which replicate autonomously, use is preferably made, depending on the host cell, of systems of the “plasmid”, “cosmid” or “minichromosome” type or systems of the viral type, the viral vectors possibly being in particular adenoviruses (Perricaudet et al., 1992), retroviruses, lentiviruses, poxviruses or herpesviruses (Epstein et al., 1992). Those skilled in the art are aware of the technology which can be used for each of these systems.

When integration of the sequence into the chromosomes of the host cell is desired, use may be made, for example, of systems of the plasmid or viral type; such viruses are, for example, retroviruses (Temin, 1986) or AAVs (Carter, 1993).

Among the nonviral vectors, preference is given to naked polynucleotides such as naked DNA or naked RNA according to the technique developed by the company VICAL, bacterial artificial chromosomes (BACs), yeast artificial chromosomes (YACs) for expression in yeast, mouse artificial chromosome (MACs) for expression in murine cells and, preferably, human artificial chromosomes (HACs) for expression in human cells.

Such vectors are prepared according to the methods commonly used by those skilled in the art, and the clones resulting therefrom can be introduced into a suitable host using standard methods, such as, for example, lipofection, electroporation, heat shock, transformation after chemical permeabilization of the membrane, or cell fusion.

The invention also comprises the host cells, in particular the eukaryotic and prokaryotic cells, transformed by the vectors according to the invention. Among the cells which can be used for the purposes of the present invention, mention may be made of bacterial cells (Olins and Lee, 1993), but also yeast cells (Buckholz, 1993), as well as animal cells, in particular mammalian cell cultures (Edwards and Aruffo, 1993), and in particular Chinese hamster ovary (CHO) cells and human cells. Mention may also be made of insect cells in which it is possible to use methods employing, for example, baculoviruses (Luckow, 1993). A preferred cellular host for expressing the proteins of the invention consists of COS cells and Hela cells.

The invention also comprises the transgenic animals, preferably mammals, except humans, comprising one of said transformed cells according to the invention. These animals can be used as models, for studying the etiology of a pathology associated with a deleterious modification of the animal homologue of the natural human RH116 protein, or for studying the effects of a viral infection caused by an RNA virus, such as HIV, on the expression of the RH116 protein in the presence or absence of an antiviral treatment, such as an RH116 antagonist, for instance murabutide for example.

Among the mammals according to the invention, animals such as rodents, in particular mice, rats or rabbits, expressing a polypeptide according to the invention, are preferred.

The transgenic animals according to the invention can overexpress the gene encoding the protein according to the invention, or their homologous gene, or express said gene into which a mutation is introduced. These transgenic animals, in particular mice, are obtained, for example, by transfection of a copy of this gene under the control of a promoter which is strong and ubiquitous, or selective for a tissue type, or after viral transcription.

Alternatively, the transgenic animals according to the invention may be made deficient for the gene encoding the polypeptide of sequence SEQ ID No. 2, or their homologous genes, by targeted inactivation by homologous recombination possibly using the LOX-P/CRE recombinase system (Rohlmann et al., 1996) or by any other system for inactivating the expression of this gene. These transgenic animals are obtained, for example, by homologous recombination on embryonic stem cells, transfer of these stem cells to embryos, selection of the chimeras affected in the reproductive lines, and growth of said chimeras.

The cells or mammals transformed as described above can also be used as models in order to study the interactions between the polypeptides according to the invention and the chemical or protein compounds involved directly or indirectly in the activities of the polypeptides according to the invention, this being in order to study the various mechanisms and interactions involved. They may in particular be used to select products which interact with the polypeptides according to the invention, in particular the protein of SEQ ID No. 2, or their variants according to the invention, as a cofactor or as an inhibitor, in particular a competitive inhibitor, or which have an agonist or antagonist activity with respect to the activity of the polypeptides according to the invention. Preferably, said transformed cells or transgenic animals are used as a model in particular for selecting compounds for decreasing the level of expression or the RNA helicase activity of the RH116 polypeptide of the invention.

In addition to their use as an analytical model, the cells and mammals according to the invention can be used in a method for producing a polypeptide according to the invention, as described below. The method for producing a polypeptide of the invention in recombinant form, itself included in the present invention, is characterized in that the transformed cells, in particular the cells of the present invention, are cultured under conditions which allow the expression and, optionally, the secretion of a recombinant polypeptide encoded by a nucleic acid sequence according to the invention, and in that said recombinant polypeptide is recovered. The recombinant polypeptides which can be obtained using this method of production are also part of the invention. They may be in glycosylated or nonglycosylated form and may or may not have the tertiary structure of the natural protein. The sequences of the recombinant polypeptides may also be modified in order to improve their solubility, in particular in aqueous solvents. Such modifications are known to those skilled in the art, such as, for example, the deletion of hydrophobic domains or the substitution of hydrophobic amino acids with hydrophilic amino acids.

These polypeptides may be produced using the nucleic acid sequences defined above, according to the techniques for producing recombinant polypeptides known to those skilled in the art. In this case, the nucleic acid sequence used is placed under the control of signals which allow its expression in a cellular host.

An effective system for producing a recombinant polypeptide requires having a vector and a host cell according to the invention. These cells can be obtained by introducing into host cells a nucleotide sequence inserted into a vector as defined above, and then culturing said cells under conditions which allow the replication and/or expression of the transfected nucleotide sequence.

The methods used for purifying a recombinant polypeptide are known to those skilled in the art. The recombinant polypeptide may be purified from cell lysates and extracts or from the culture medium supernatant, by methods used individually or in combination, such as fractionation, chromatography methods, immunoaffinity techniques using specific monoclonal or polyclonal antibodies, etc. A preferred variant consists in producing a recombinant polypeptide fused to a “carrier” protein (chimeric protein). The advantage of this system is that it allows stabilization and a decrease in proteolysis of the recombinant product, an increase in solubility during renaturation in vitro and/or simplification of the purification when the fusion partner has affinity for a specific ligand.

The polypeptides according to the present invention can also be obtained by chemical synthesis using one of the many known forms of peptide synthesis, for example the techniques using solid phases (see in particular Stewart et al., 1984) or techniques using partial solid phases, by fragment condensation or by conventional synthesis in solution. The polypeptides obtained by chemical synthesis and which may comprise corresponding unnatural amino acids are also included in the invention.

The invention also relates to a monoclonal or polyclonal antibody and its fragments, characterized in that they selectively and/or specifically bind a polypeptide according to the invention. The chimeric antibodies, the humanized antibodies and the single-chain antibodies are also part of the invention. The antibody fragments according to the invention are preferably Fab, F(ab′)2 or Fv fragments.

The polypeptides according to the invention make it possible to prepare monoclonal or polyclonal antibodies. The monoclonal antibodies may advantageously be prepared from hybridomas according to the technique described by Kohler and Milstein in 1975.

The polyclonal antibodies may be prepared, for example, by immunizing an animal, in particular a mouse, with a polypeptide according to the invention combined with an adjuvant of the immune response, and then purifying the specific antibodies contained in the serum of the immunized animals, on an affinity column to which the polypeptide which was used as antigen has been attached beforehand. The polyclonal antibodies according to the invention may also be prepared by purification on an affinity column, on which a polypeptide according to the invention has been immobilized beforehand.

According to a particular embodiment of the invention, the antibody is capable of inhibiting the interaction between the RH116 polypeptide and the RNA sequence to which this binds in order to impair the physiological function of said RH116 polypeptide.

The invention also relates to methods for detecting and/or purifying a polypeptide according to the invention, characterized in that they use an antibody according to the invention. The invention also comprises purified polypeptides, characterized in that they are obtained using a method according to the invention.

Moreover, besides their use for purifying the polypeptides, the antibodies of the invention, in particular the monoclonal antibodies, may also be used for detecting these polypeptides in a biological sample.

For these various uses, the antibodies of the invention may also be labeled in the same way as described previously for the nucleic acid probes of the invention, and preferably with labeling of the enzymatic, fluorescent or radioactive type.

The antibodies of the invention also constitute a means for analyzing the expression of a polypeptide according to the invention, for example using immunofluorescence, gold labeling and/or enzymatic immunoconjugates. More generally, the antibodies of the invention may advantageously be used in any situation where the expression of a polypeptide according to the invention must be observed, and more particularly in immunocytochemistry, in immunohistochemistry or in Western blotting experiments.

They may in particular make it possible to demonstrate abnormal expression of these polypeptides in biological specimens or tissues.

More generally, the antibodies of the invention may advantageously be used in any situation where the expression of a polypeptide according to the invention, which may be normal or mutated, must be observed. Thus, a method for detecting a polypeptide according to the invention, in a biological sample, comprising the steps of bringing the biological sample into contact with an antibody according to the invention and of demonstrating the antigen-antibody complex formed, is also a subject of the invention.

Also falling within the context of the invention is a kit of reagents for detecting and/or assaying a polypeptide according to the invention, in a biological sample, characterized in that it comprises the following elements: (i) a monoclonal or polyclonal antibody as described above; (ii) where appropriate, the reagents for constituting the medium suitable for the immunoreaction; (iii) the reagents for detecting the antigen-antibody complexes produced by the immunoreaction. This kit is in particular of use for carrying out Western blotting experiments; these experiments make it possible to study the regulation of expression of the polypeptide according to the invention using tissues or cells. This kit is also of use in immunoprecipitation experiments for demonstrating in particular the proteins which interact with the polypeptide according to the invention. This kit is also of use for detecting and/or assaying a polypeptide according to the invention using a method which involves the ELISA technique, immunofluorescence, radioimmunology (RIA technique) or an equivalent technique.

The invention also comprises a method for detecting and/or assaying a polynucleotide according to the invention, in a biological sample, characterized in that it comprises the following steps: (i) isolating the DNA from the biological sample to be analyzed, or obtaining a cDNA from the RNA of the biological sample; (ii) specifically amplifying the DNA encoding the polypeptide according to the invention using primers; (iii) analyzing the amplification products. An object of the invention is also to provide a kit for detecting and/or assaying a nucleic acid according to the invention, in a biological sample, characterized in that it comprises the following elements: (i) a pair of nucleic acid primers according to the invention, (ii) the reagents required to carry out a DNA amplification reaction and, optionally, (iii) a component for verifying the sequence of the amplified fragment, more particularly a probe according to the invention.

The invention also comprises a method for detecting and/or assaying nucleic acid according to the invention, in a biological sample, characterized in that it comprises the following steps: (i) bringing a polynucleotide according to the invention into contact with a biological sample; (ii) detecting and/or assaying the hybrid formed between said polynucleotide and the nucleic acid of the biological sample. A subject of the invention is also to provide a kit for detecting and/or assaying nucleic acid according to the invention, in a biological sample, characterized in that it comprises the following elements: (i) a probe according to the invention, (ii) the reagents required to carry out a hybridization reaction and/or, where appropriate, (iii) a pair of primers according to the invention, and also the reagents required for a DNA amplification reaction.

Preferably, the biological sample according to the invention in which the detection and the assaying are carried out consists of a body fluid, for example a human or animal serum, or blood, or of biopsies.

The methods for determining an allelic variability, a mutation, a deletion, a loss of heterozygocity or any genetic abnormality of the gene encoding the polypeptide according to the invention, characterized in that they use a nucleic acid sequence, a polypeptide or an antibody according to the invention, are also part of the invention.

It is possible to detect these mutations directly by analyzing the nucleic acid and the sequences according to the invention (RNA or cDNA), but also via the polypeptides according to the invention. In particular, the use of an antibody according to the invention which recognizes an epitope carrying a mutation makes it possible to distinguish between a “healthy” protein and a protein “associated with a pathology”.

This method of diagnosis and/or of prognostic assessment may be used preventively, or so as to serve in establishing and/or confirming a clinical condition in a patient. The analysis may be carried out by sequencing all or part of the gene (i.e. the exons), or by other methods known to those skilled in the art. Methods based on PCR, for example PCR-SSCP, which makes it possible to detect point mutations, may in particular be used. The analysis may also be carried out by attachment of a probe according to the invention to a DNA chip containing at least one polynucleotide according to the invention and hybridization on these microplates. A DNA chip containing a sequence according to the invention is also one of the subjects of the invention.

Similarly, a protein chip containing an amino acid sequence according to the invention is also a subject of the invention. Such a protein chip makes it possible to study the interactions between the polypeptides according to the invention and other proteins or chemical compounds, and may thus be of use in screening compounds which interact with the polypeptides according to the invention. The protein chips according to the invention may also be used for detecting the presence of antibodies against the polypeptides according to the invention in the serum of patients. A protein chip containing an antibody according to the invention may also be used.

Substances may also be tested and identified for their ability to modulate the enzymatic activities of the polypeptide of the invention. The expression “a substance which modulates an enzymatic activity” is intended to denote a substance which changes the enzymatic activity compared with the enzymatic activity measured in the absence of the substance to be tested. For example, such a substance may partially or totally inhibit the RNA helicase activity; such an activity may be measured by methods known to those skilled in the art. For example, synthetic oligonucleotides can be immobilized on a matrix and hybridized with a labeled complementary oligoribonucleotide. The hybridized oligoribonucleotides are then used with a polypeptide of the invention, which releases a certain measurable amount of the labeled oligoribonucleotide, not attached to the matrix, given its RNA helicase activity. The effects of the presence or absence of potential modulators of the RH116 RNA helicase of the invention, or one of its fragments, can thus be tested. Another alternative consists in using the protocol described by Jaramillo et al. (1991); this method consists in mixing a ³²P-labeled duplex RNA substrate with the polypeptide of the invention in a buffered solution; the reaction is stopped by adding a mixture of glycerol/SDS/EDTA/bromophenol blue; the mixture is then prepared on an SDS-PAGE gel (8%) according to standard conditions. The RNA helicase activity is estimated by the ratio of the amount of monomeric RNA to the amount of duplex RNA. Other protocols are also available (Rozen et al. (1990); Pause et al. (1992); Lain et al. (1993), Lee and Hurwitz (1993)). These assays can be carried out in microplates in order to simultaneously test large amounts of modulators and/or inhibitors of the polypeptide according to the invention.

Such substances may be developed and selected beforehand, by molecular modeling, in order to identify substances liable to react with a polypeptide according to the invention. It is possible to identify a substance which affects the ability of the protein of the invention to bind to ATP or other substrates, such as RNA, DNA or RNA/protein complexes. The substrates which interfere with a substrate binding site of the protein of the invention, or with a site which affects such functional epitopes, can be identified using methods known to those skilled in the art (for review see Fruehleis et al. (1987); Perun et al. (1989); Van de Waterbeemd (1994), Blundell (1996)).

The invention therefore relates to a method for screening a compound capable of affecting the level of cellular expression and/or the RNA helicase activity of an RH116 polypeptide according to the invention, which comprises the following steps of (i) bringing a cell chosen from the host cell of the invention and a eukaryotic cell, preferably a human cell, expressing or containing the polypeptide according to the invention, into contact with one or more potential compounds or ligands capable of penetrating or being introduced into said cell, and (ii) detecting and/or measuring the level of cellular expression and/or the RNA helicase activity. The gene encoding the polypeptide according to the invention which is present in the host cell or in a eukaryotic cell, preferably a human cell, corresponds at least to a polynucleotide sequence encoding the polypeptide of the invention, preferably in the form of genomic DNA or of cDNA, functionally linked to the promoter sequence of the human RH116 gene or of the homologous gene of an animal species such as the mouse. The compounds screened using such a method and capable of affecting the level of expression of the RH116 RNA helicase are preferably compounds capable of interacting with the regulatory polynucleotide sequences (promoter, upstream sequence, enhancer, silencer, insulator, etc.) of the gene naturally encoding the polypeptide according to the invention, or the compounds capable of interacting with transcription factors (general transcription factors or tissue-specific factors) involved in regulating the transcription of the gene encoding the polypeptide according to the invention, so as to form a complex capable of affecting the transcription of the gene encoding the RH116 polypeptide of the invention, i.e. of increasing, of decreasing, of modulating or of eliminating the transcription of said gene. The techniques for detecting and/or measuring transcriptional activity are known to those skilled in the art. Mention should in particular be made of Northern blotting and RT-PCR technology, which can be employed with the polypeptides of the invention used as probes or as primers, respectively.

The invention also relates to a method for screening a compound capable of affecting the RNA helicase activity of a polypeptide according to the invention, which comprises the following steps of (i) bringing said polypeptide into contact with one or more potential compound(s) or ligand(s), in the presence of reagents required for implementing the RNA helicase activity, and (ii) detecting and/or measuring the RNA helicase activity.

According to a preferred embodiment, the methods for screening compounds described above are characterized in that said screened compound decreases or destroys the level of expression and/or the RNA helicase activity of the RH116 polypeptide of the invention. The compound which can be obtained using the methods described above and which decreases the level of expression and/or the RNA helicase activity of the RH116 polypeptide of the invention is an RH116 antagonist and also constitutes one of the subjects of the invention; this compound is characterized in that it is chosen from a polynucleotide according to the invention used as an antisense nucleic acid sequence, an antisense expression vector according to the invention, an antibody according to the invention, and muramyl peptides, preferably murabutide, or from any other antagonist of the polypeptide according to the invention.

In fact, partially or totally blocking the biological activity of the RH116 polypeptide of the invention and of its fragments constitutes a means of inhibiting or destroying viral replication. Specifically, the unspliced genomic RNA and the incompletely spliced mRNA of retroviruses such as HIV need to be exported into the cytoplasm for packaging and/or translation. Such a process is mediated either by a CIS-acting constitutive transport element (CTE) for simple retroviruses, or by the Rev viral protein acting in TRANS with responsive elements (Rev Responsive element for RRE) for complex retroviruses such as HIV. The RH116 RNA helicase according to the invention is capable of constituting a cofactor for the CTE and playing a role in RRE-mediated gene expression and also of playing a role in HIV replication.

The present invention therefore proposes to provide compounds capable of partially or totally blocking the RNA helicase activity of the polypeptide of the invention in order to decrease or destroy viral replication. Among these, mention should be made of a polynucleotide according to the invention used as an antisense nucleic acid sequence, an antisense expression vector according to the invention, an antibody according to the invention, muramyl peptides, preferably murabutide, or any other antagonist of the polypeptide according to the invention. Preferably, the muramyl peptides and murabutide constitute RH116 antagonists in the cells of HIV+ patients.

The present invention is not limited to only the HIV virus, but to all viruses directly or indirectly involving an RNA helicase in their replication. The term “viruses” is intended to denote enveloped or nonenveloped, single-stranded or double-stranded, DNA or RNA viruses. Preferably, the viruses belong to the family Retroviridae, Orthomyxoviridae, Rhabdoviridae, Bunyaviridae, Adenoviridae, Hepadnaviridae, Herpesviridae or Poxviridae. Preferably, the hepadnaviruses are the hepatitis B and hepatitis C viruses.

The biological activity of the polypeptide of the invention is not restricted only to post-transcriptional control of viral RNAs, but also contributes to the transcriptional control of RNAs in general. Thus, the polypeptide of the invention constitutes a therapeutic target of interest for compounds capable of impairing the RNA helicase activity involved in other normal or pathological biological processes.

RNA helicase constitutes a target of choice for compounds intended for the treatment of cancer. In fact, various articles in the scientific literature refer to the involvement of RNA helicases in tumorigenesis. Thus, overexpression of the DEAD-box1 (DDX1) protein may play a role in the progression of tumors such as neuroblastoma (Nb) and retinoblastoma (Godbout et al. 1998) by impairing the normal secondary structure and the level of expression of the RNAs of cancer cells. Other RNA helicases have been directly or indirectly implicated in tumorigenesis. Thus, the murine p68 protein is mutated in tumors induced by ultraviolet light; the DDX6 RNA helicase gene is located at the point of chromosomal breaking associated with B-cell lymphoma. Similarly, a chimeric protein comprising DDX10 and the nucleoporin NUP98 appears to be involved in the pathogenesis of certain myeloid diseases. The compounds capable of decreasing or destroying the RNA helicase activity of RH116 therefore constitute compounds of interest intended for the preventive or curative treatment of cancer. Among the cancers which can be treated with the RH116-antagonist compounds, mention should be made, in a non [lacuna] manner of cancers such as adenocarcinoma, leukemia, lymphoma, melanoma, myeloma, sarcoma, glioma or teratocarcinoma, and more particularly cancer of the adrenal gland, bladder cancer, bone cancer, cancer of the marrow, breast cancer, cancer of the gastro-intestinal tract, liver cancer, lung cancer, pancreatic cancer, ovarian cancer, cancer of the uterus, testicular cancer, prostate cancer and throat cancer.

Similarly, the compounds capable of decreasing or eliminating the RNA helicase activity of RH116 therefore constitute compounds of interest intended for the preventive or curative treatment of other pathologies, such as rheumatism, hereditary diseases, arthritis, artherosclerosis, osteoporosis, acute and chronic infectious diseases, autoimmune diseases, diabetes, and also problems associated with organ rejection in transplantation. More particularly, the invention relates to the immune and autoimmune diseases which also include AIDS (acquired immunodeficiency syndrome).

The inhibitors, antagonists and other compounds capable of decreasing or eliminating the RNA helicase activity of RH116 constitute compounds of interest for the preventive or curative treatment of autoimmune diseases. Specifically, it has recently been reported by Takeda et al. (1999) that RNA helicase A acts as an autoantigen in patients suffering from systemic lupus erythematosus. Thus, the polynucleotide according to the invention used as an antisense nucleic acid sequence, the antisense expression vector according to the invention, the antibody according to the invention, the muramyl peptides, and preferably murabutide, or any other antagonist of the RH116 polypeptide according to the invention can be used for the treatment of autoimmune diseases. Among autoimmune diseases, mention should be made more particularly of uveitis, Bechet's disease, sarcoidosis, Sjögren's syndrome, rheumatoid arthritis, juvenile arthritis, Fiessinger-Leroy-Reiter's syndrome, gout, osteoarthrosis, systemic lupus erythematosus, acute disseminated lupus erythematosus, polymyositis, myocarditis, primary biliary cirrhosis, Crohn's disease, ulcerative colitis, multiple sclerosis and other demyelinating diseases, aplastic anemia, thrombocytopenic purpura, multiple myeloma and B-lymphocyte lymphoma, Simmonds' disease panhypopituitarism, Basedow-Graves' disease and Graves' ophthalmopathy, subacute thyroiditis and Hashimoto's disease, Addison's disease, insulin dependent diabetes mellitus (type 1), Addison's disease, adult respiratory distress syndrome, allergies, anemia, asthma, autoimmune hemolytic anemia, bronchitis, atopic dermatitis, emphysema, and episodic lymphopenia.

According to another embodiment of the invention, the method for screening a compound of the invention is characterized in that said screened compound increases the level of expression and/or the RNA helicase activity of the RH116 polypeptide of the invention. The compound thus screened, which is also a subject of the invention, constitutes an agonist of the polypeptide according to the invention. Among the agonist compounds which can be obtained using the method according to the invention, mention should be made of muramyl peptides, and preferably murabutide; specifically, the inventors have demonstrated experimentally that the latter compound, murabutide, increases the level of expression of RH116 in the cells of healthy donors, thus behaving as an agonist of the polypeptide of the invention.

According to another embodiment, the invention relates to a method for screening compounds capable of affecting the functional activity of a polypeptide according to the invention. Such a method comprises the steps of (i) bringing said polypeptide into contact with one or more potential ligand(s), in the presence of reagents required to carry out a reaction chosen from the nuclear and/or mitochondrial RNA splicing reaction, RNA editing reaction, rRNA processing reaction, translation initiation reaction, reaction of nuclear mRNA export to the cytoplasm and mRNA degradation reaction, and (ii) detecting and/or measuring said reaction. The screened compound which can be obtained using the method also falls within the scope of this invention.

The invention therefore relates to a compound, characterized in that it is chosen from an antibody according to the invention, a polypeptide according to the invention, a polynucleotide according to the invention, an oligonucleotide according to the invention, a vector according to the invention, an antisense expression vector according to the invention, a cell according to the invention, and a compound which can be obtained using the various screening methods according to the invention, as a medicinal product, and in particular as active principles of a medicinal product; these compounds will preferentially be in soluble form, combined with a pharmaceutically acceptable vehicle. The expression “pharmaceutically acceptable vehicle” is intended to denote any type of vehicle conventionally used in the preparation of injectable compositions, i.e. a diluent or a suspending agent, such as an isotonic or buffered saline solution. Preferably, these compounds will be administered systemically, in particular intravenously, intramuscularly, intradermally or orally. Their optimal methods of administration, doses and pharmaceutical forms can be determined according to the criteria generally taken into account in establishing a treatment suitable for a patient, such as, for example, the age or body weight of the patient, the seriousness of his or her general condition, the tolerance to the treatment and the side effects observed, etc.

Preferably, the compounds of the invention as medicinal products are intended for the prevention and/or treatment of pathologies selected from the group composed of cancer, acute or chronic infectious diseases such as infections with HIV or the hepatitis B or C virus, hereditary genetic diseases, immune and autoimmune diseases, rheumatism, arthritis, artherosclerosis, osteoporosis and diabetes, and for the prevention of organ transplant rejection.

Among the compounds as medicinal products of the invention, the RH116 polypeptide-antagonist compounds are particularly preferred for preparing a medicinal product intended for the treatment of viral pathologies such as acquired immunodeficiency syndrome (AIDS) or hepatitis.

One of the objects of the present invention is also to provide a pharmaceutical composition for preventive and curative treatment of viral pathologies, and in particular of AIDS or hepatitis, characterized in that it contains a therapeutically effective amount of an RH116 polypeptide-antagonist compound and of a pharmaceutically acceptable vehicle. More particularly, the invention is also directed toward providing a product comprising at least one RH116-antagonist compound and at least one other antiviral agent, as a combination product for use simultaneously, separately, or spread out over time in antiviral therapy, preferably anti-HIV therapy. This other antiviral agent is preferably chosen from (i) nucleotide or non-nucleotide inhibitors of reverse transcriptase, such as, for example, 3′-azido-3′-deoxythymidine (AZT), 2′,3′-dideoxyinosine (ddI), 2′,3′-dideoxycytidine (ddC), (−)2′,3′-dideoxy-3′-thiacytidine (3TC), 2′,3′-didehydro-2′,3′-dideoxythymidine (d4T), (−)2′-deoxy-5-fluoro-3′-thiacytidine (FTC), TIBO, HEPT, TSAO, α-APA, nevirapine, BAHP or phosphonoformic acid (PFA), and (ii) viral protease inhibitors such as indinavir and saquinavir.

According to another embodiment, it is also within the scope of the invention to provide a method of therapeutic or prophylactic treatment of a disease associated with an increase in the expression or the activity of the RH116 polypeptide according to the invention. This method comprises administering a therapeutically effective amount of an antagonist of the polypeptide of the invention to a patient who requires such a treatment.

The invention also relates to the use of a polypeptide according to the invention, of a polynucleotide according to the invention and/or of an RH116 polypeptide-agonist compound according to the invention, for preparing a medicinal product intended for the prophylactic treatment of pathologies. Specifically, vaccines generally induce immunity to an infection or to a disease by generating, in a patient, an immune response against a specific antigen associated with the infection or with the disease. However, in many cases, purified antigens are poor immunogens. In such cases, immunomodulating agents such as an adjuvant or an immunostimulant must be used to increase the immune response. However, one of the disadvantages of many adjuvants currently used is their toxicity. There is therefore a real need to identify novel compounds which make it possible to increase specific immune responses; this is what the present invention proposes to do. Specifically, the polypeptides and the polynucleotides of the invention and the RH116 polypeptide-agonist compounds according to the invention can be used to prepare a medicinal product intended to cause or to increase the immune response to a vaccine in a patient.

Other characteristics and advantages of the invention are apparent in the remainder of the description with the examples represented below. In these examples, reference will be made to the following figures.

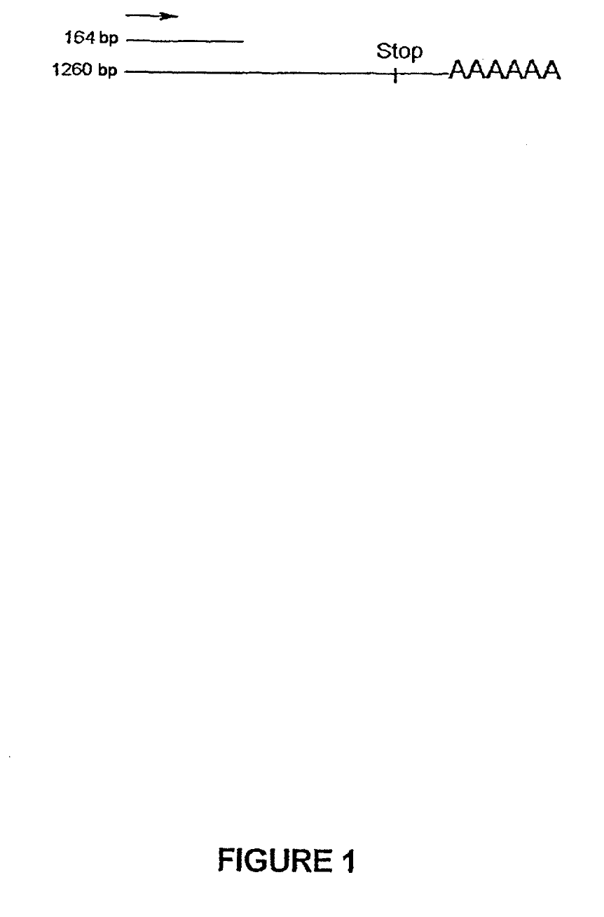

FIG. 1: Strategy used to obtain the 3′ end of the cDNA encoding RH116

The upper short fragment is the initial fragment of 164 bp obtained by DD-RT PCR and corresponds to the 3′ region of the cDNA encoding the RH116 polypeptide of the invention. This longer fragment of 1 260 bp was obtained after a 3′ RACE (lower line).

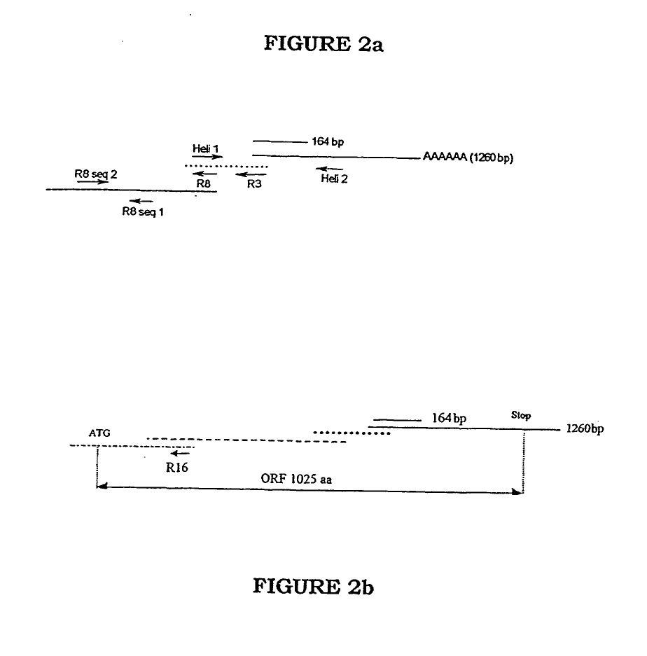

FIG. 2: Strategy for obtaining the 5′ region of the cDNA encoding RH116. (A) The portion of the 5′ end was obtained after two PCR reactions (5′RACE for Rapid Amplification of cDNA Ends), the fragments obtained are symbolized by ( . . . ) (R3) and by ( - - - ) (R8). This strategy therefore allowed the inventors to obtain an open reading frame (ORF) of 853 amino acids. The oligonucleotides HELI 1 and HELI 2, which will serve as primers for the RT-PCRs, are indicated. (B) The 5′-terminal portion of the cDNA corresponding to the RH116 polypeptide was obtained after a PCR reaction using the primer R16 (5′RACE). The 946 bp fragment obtained is symbolized as a broken line ( - - - ).

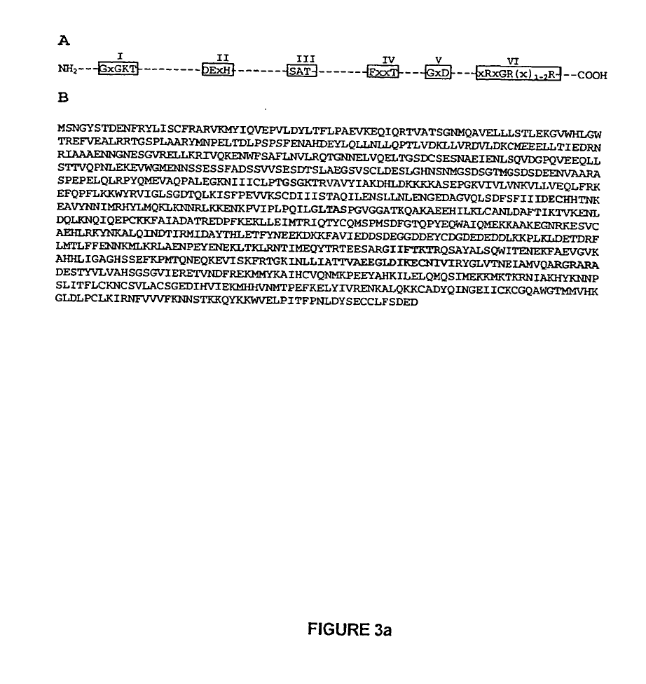

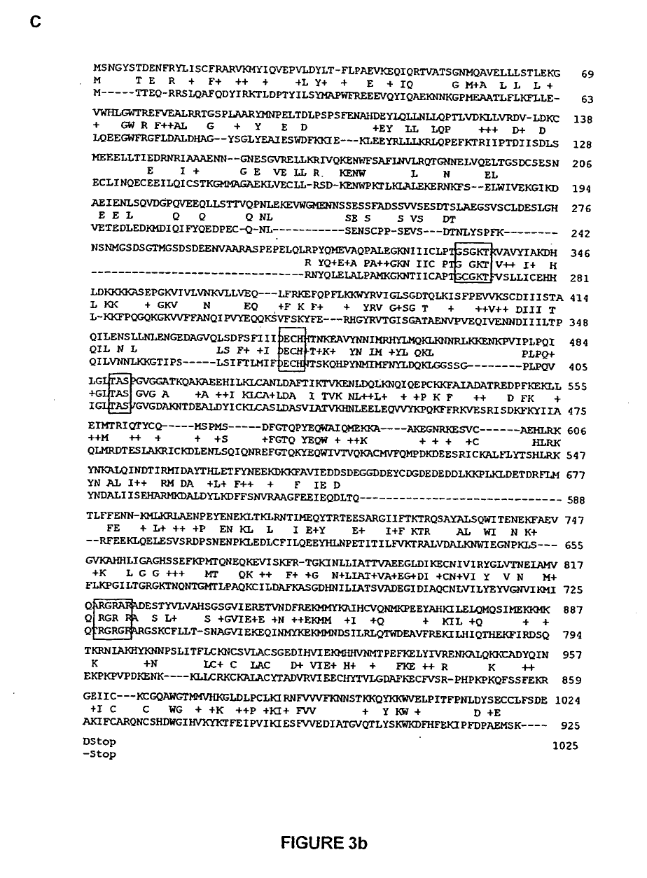

FIG. 3: Amino acid sequence of the putative human RNA helicase RH116 and comparison with the putative RNA helicase (RIG-1). (A) Diagrammatic representation of the highly conserved regions of amino acids characteristic of the putative ATP-dependent RNA helicases of the “DEAD-BOX” protein family. The conserved motif is boxed (the “x” indicates any amino acid), in a SAT motif, -A- is the highly conserved amino acid, S and T can be replaced with any amino acid in any individual case (Luking et al., 1998). In motif VI, (X)₁₋₂ indicates any of the 2 amino acids. (B) Amino acid sequence of the RH116 protein deduced from the cDNA sequence (EMBL accession No. AY017378). Six conserved motifs characteristic of the “DEAD-BOX” protein family are indicated by letters in bold. (C) Sequence alignment of the 1 025 aa open reading frame (clone 10.5) (upper sequence) with a human RNA helicase (RIG-1) described by Y. W. SUN (accession No. AF 038963) (lower sequence). The sequence homologies are given in the intermediate line between the two sequences. The boxed sequences correspond to the RNA helicase conserved domains; domains G-GKT and DEXH correspond to the ATPase domains and domains SAT (-A-) and RGR—R (GR—R), which are specific for RNA helicases, are responsible for the binding and unfolding of the target RNA.

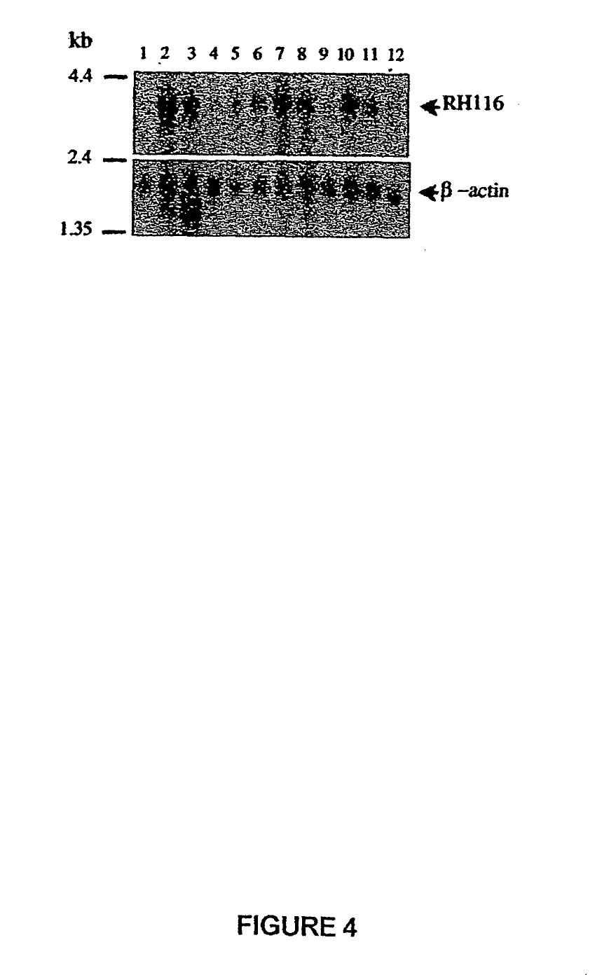

FIG. 4: Northern blotting analysis of expression of the RH116 messenger RNA in many human tissues (CLONTECH). The polyA+ RNA obtained from brain (1), heart (2), skeletal muscle (3), colon (4), thymus (5), spleen (6), kidney (7), liver (8), small intestine (9), placenta (10), lung (11), and peripheral blood leukocytes (12) was hybridized with an oligonucleotide probe derived from the RH116 messenger RNA and labeled at its ends with ³²P phosphorus (upper part of the figure). Equivalent amounts of polyA+ RNA are also hybridized with a β-actin probe used as a control (lower part of the figure). Both in heart and skeletal muscle, two forms of β-actin messenger RNA exist, a 2 kb form and a 1.6-1.8 kb form. This difference in size is not due to degradation of the messenger RNA, but to hybridization of the probe with the a form or the γ form of the actin (see instructions from the manufacturer CLONTECH).

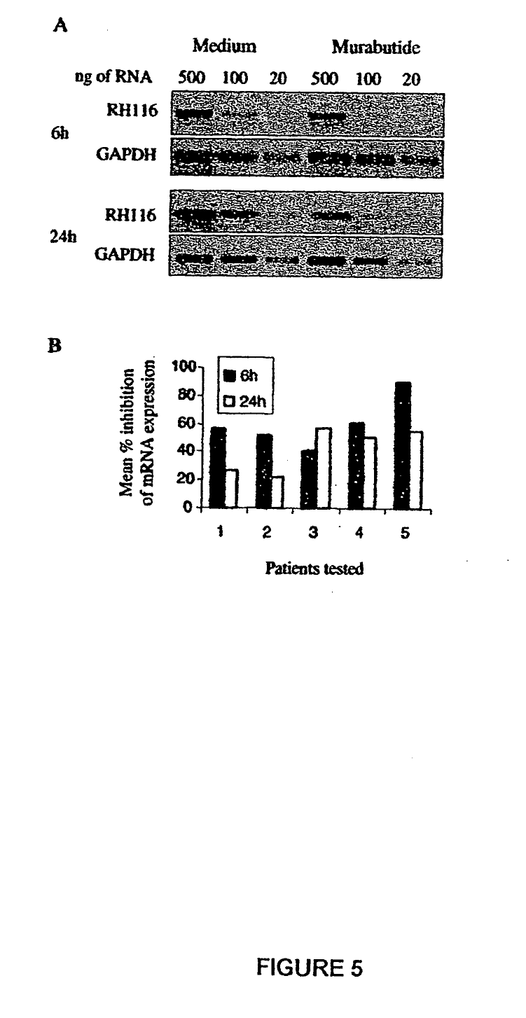

FIG. 5: Inhibition of the expression of the RH116 messenger RNA induced by murabutide in endogenously infected and CD8-depleted PBMCs. PBMCs activated with PHA and CD8-depleted are maintained in culture in the absence (medium) or presence of murabutide at 10 μg/ml for 6 or 24 hours. (A) RNA samples (20, 100 and 500 ng) are subjected to amplification by RT PCR with a pair of primers specific for detecting RH116 messenger RNA. The GAPDH messenger RNA (internal control) expressed constitutively is also amplified in the same samples. (B) The mean percentage inhibition of expression of the RH116 messenger RNA is observed in samples from 5 patients infected with HIV-1.

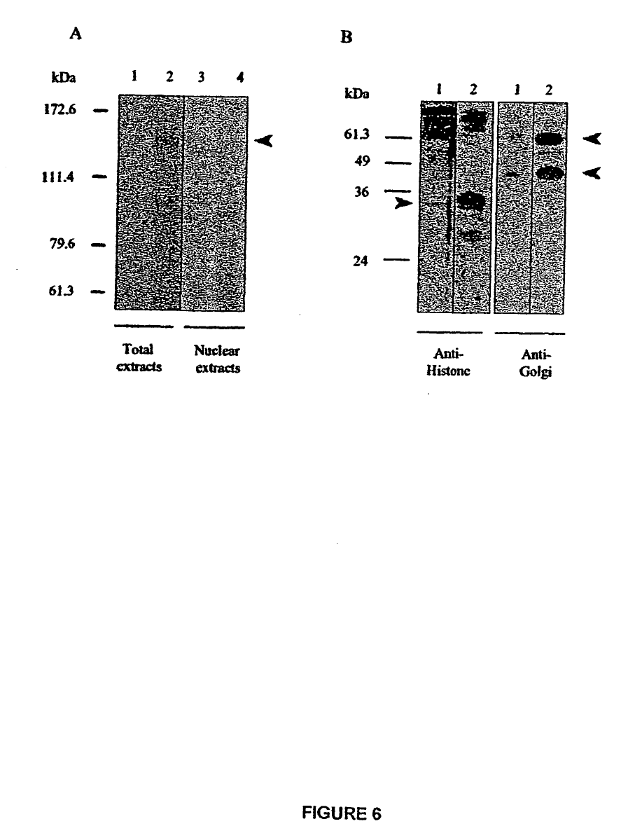

FIG. 6: Immunoprecipitation of the RH116 protein from cell extracts labeled with ¹²⁵I iodine obtained from U937 cells. (A) Total cell extracts (1, 2) or nuclear extracts (3, 4) are labeled and immuno-precipitated using 20 μg of immunoglobulins purified from normal serum (1, 3) or from murine serum obtained against the RH116₁₋₃₃₅ protein (2, 4). The samples are loaded onto an 8% SDS-PAGE gel. (B) The quality of the cell fractionation is verified by Western blotting analysis using an antibody against the 58 kDa protein of the Golgi apparatus (cytoplasmic protein) and an anti-histone 1 (nuclear protein) antibody in total extracts (1) and/or nuclear extracts (2).

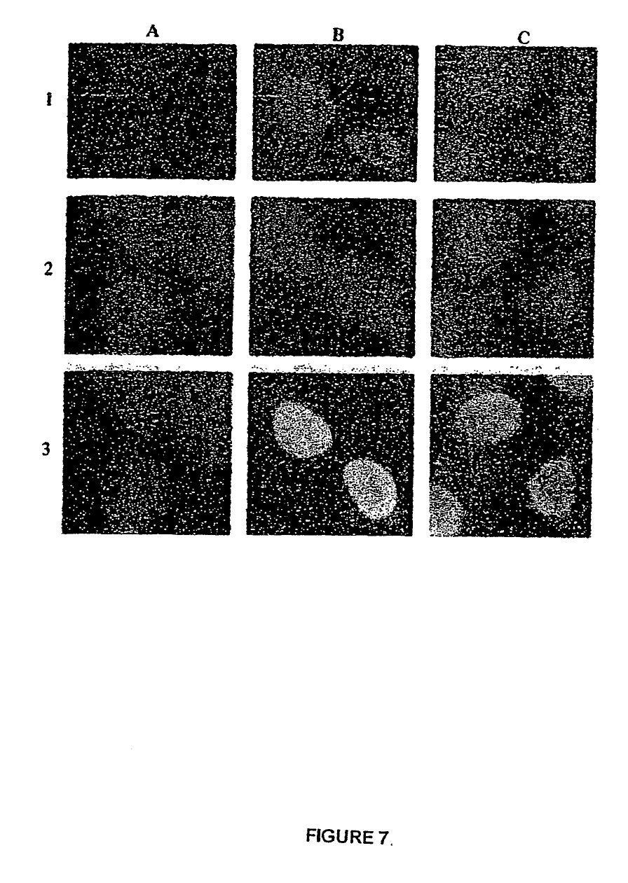

FIG. 7: Immunolocalization of the RH116 protein in HeLa-CD4+ cells. Methanol-fixed Hela-CD4+ cells are incubated with normal murine serum (A), anti-histone H1 monoclonal antibodies (Santa Cruz) (B) or a murine antiserum against the RH116₁₋₃₃₅ protein (C), and are stained with anti-mouse goat IgG antibodies conjugated to FITC and/or with propidium iodide. The immunofluorescence shows green FITC staining (1), red nuclear counterstaining corresponding to the propidium iodide (2) and double staining (3).

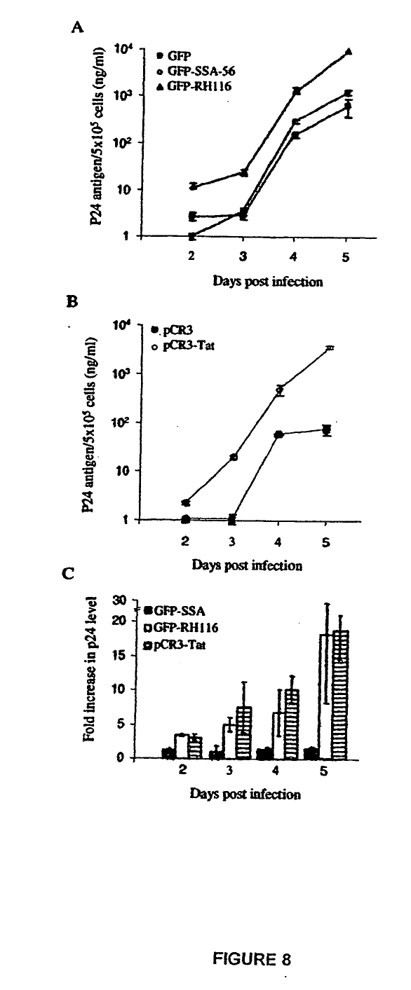

FIG. 8: Expression of the viral antigen P24 by transfected HeLa CD4+ cells infected with the HIV-1_{LAI} virus. The HeLa-CD4+ cells are transiently transfected, firstly, with the plasmid pEGFP (GFP) or with the plasmid pEGFP into which are respectively cloned the cDNAs of RH116 or of SSA (GFP-RH116 and GFP-SSA respectively) and, secondly, with the plasmid pCR3 or the plasmid pCR3 into which is cloned Tat (pCR3-Tat); the cells are infected 24 hours later with the HIV-1_{LAI} virus. From the 2nd day after infection of the cells, the culture media are collected daily until the 5th day.

Viral replication is evaluated by the release of the P24 antigen by the cells (cell concentration standardized at 5×10⁵ cells/ml) into the culture supernatants of cells transfected with GFP, GFP-RH116 or GFP-SSA (A) and of cells transfected with pCR3 or pCR3-Tat (B). (C) Increase in the P24 antigen in the cells infected, and transfected with GFP-RH116, GFP-SSA or Tat, compared with the respective corresponding controls (GFP or pCR3 respectively). The results corresponding to the mean values plus or minus the standard deviation of the values obtained for 5 (GFP-RH116 and GFP-SSA) and for 3 independent experiments (pCR3-Tat).

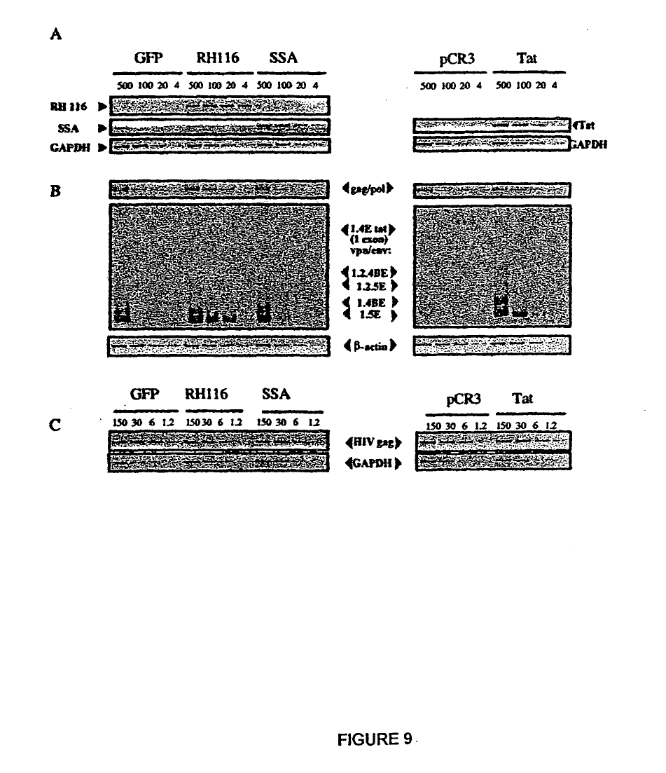

FIG. 9: Semi-quantitative RT PCR representative of the expression of the viral DNA and of the messenger RNA in transfected HeLa-CD4+ cells infected with the HIV-1_{LAI} virus. The RNA samples (500, 100, 20 and 4 ng) are subjected to RT PCR amplifications with specific primers in order to detect overexpression of the mRNAs encoding the RH116, SSA-56 or TAT protein (A). The RNA samples are subjected to RT PCR amplification in order to detect the viral mRNA (B) with a pair of primers GAG04-GAG06 in order to detect the unspliced GAG or POL mRNAs and with a pair of primers BSS/KPNA in order to detect the viral transcripts of intermediate size or which are singly spliced. These messenger RNAs are named on the basis of the exons which they contain and of the proteins which they produce (Neuman, 1994). (C) The total DNA is extracted 24 hours after infection and varying concentrations (150, 30, 6 and 1.2 ng) are used as a matrix for a PCR amplification using a pair of primers GAG04-GAG06 in order to detect the HIV-1 GAG gene. The cellular equivalent is determined by amplification of the β-actin or GAPDH genes.

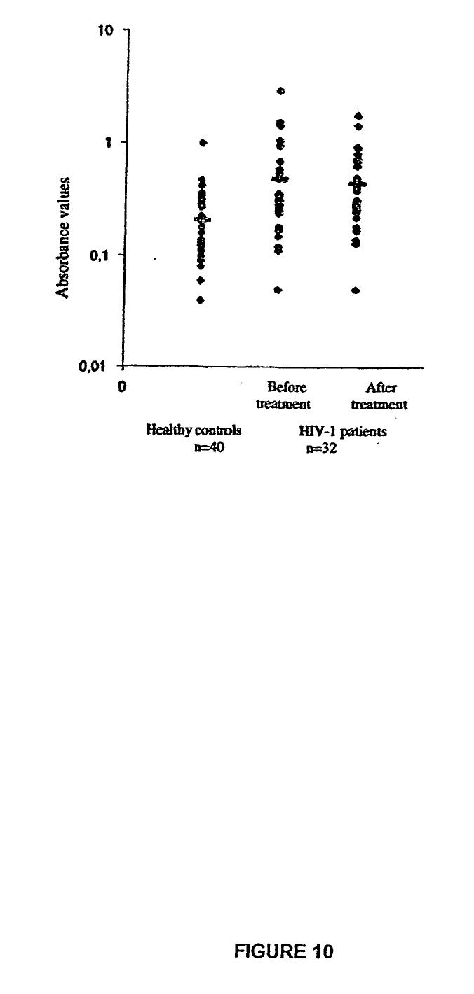

FIG. 10: Detection of autoantibodies against the RH116 protein in healthy control patients and patients infected with HIV before and after antiviral treatment. The horizontal bars reflect the arithmetic mean values.

### EXAMPLES

### Materials and Methods

### 1. Cells and Culturing Conditions

The HeLa-CD4+ cells (provided by Doctor HUBER, LABORATOIRE DE VIROLOGIE [LABORATORY OF VIROLOGY], Lille, France) and the U937 cells are cultured, respectively, in DMEM medium or in RPMI 1640 medium supplemented with 10% of heat-inactivated fetal calf serum (LIFE TECHNOLOGIES-INVITROGEN, Cergy-Pointoise, France). 2 mM L-glutamine (LIFE TECHNOLOGIES-INVITROGEN, Certy-Pontoise, France) and 1% of gentamycin (SHERING-PLOUGH, Levallois-Perret, France).

### 2. Preparation of Human Peripheral Blood Mononuclear Cells (PBMCs)

Venous blood is collected from patients sero-positive for HIV-1 and peripheral blood mononuclear cells (PBMCs) are isolated by centrifugation on Ficoll-Hypaque (AMERSHAM PHARMACIA BIOTECH, Uppsala, Sweden). The cells are washed carefully in order to remove the platelets and are depleted of CD8+ cells using electro-magnetic beads covered with an anti-CD8 antibody (DYNAL, Oslo, Norway). The depleted PBMCs obtained using the manufacturer's protocol contain less than 3% of CD8+ cells after analysis by flow cytometry. The cells are then cultured in RPMI 1640 supplemented with 10% of heat-inactivated AB serum (ETABLISSEMENT DE TRANSFUSION SANGUINE [BLOOD TRANSFUSION ESTABLISHMENT], Lille, France) and then stimulated with phyto-hemagglutinin (PHA) (5 μg/ml for 3 days). Next, the PBMCs are cultured and then recovered in the presence or absence of murabutide (N-acetylmuramyl-L-alanyl-D-glutamine-n-butyl ester) (provided by ISTAC SA, Lille, France) (10 μg/ml) and interleukin 2 (IL-2) (10 μg/ml) for 6 or 24 hours.

### 3. “Differential display-RT-PCR” (DD-RT-PCR) Experiment

The DD-RT-PCR was carried out on total RNA extracts of PHA-activated and CD8-depleted PBMCs obtained from HIV-1 patients, as previously described (Liang and Pardee, 1992, with some modifications). Briefly, 1 mg of total RNA is incubated at 75° C. for 10 minutes in the presence of 2 mM of oligo (dT) primers, T₁₂BA (B being a mixture of G, T and C), T₁₂VT (V being a mixture of A, G and C), T₁₂HG (H being a mixture of A, C and T), T₁₂DC (D being a mixture of A, G and T), and ribonuclease-free water, in a final volume of 11 μl. The complementary DNA (cDNA) is then synthesized using RNAse H Superscript reverse transcriptase (LIFE TECHNOLOGIES-INVITROGEN, Cergy-Pointoise, France) in 25 μl containing 10 mM of dithiotreitol and 20 μm of dNTP for one hour at 37° C., before denaturation for 10 min at 95° C. The cDNA synthesized is then amplified with one unit of Ampli Taq Gold polymerase (APPLIED BIOSYSTEMS, Foster City, Calif., United States), 2.5 mM of MgCl₂, 2 μM of dNTP and 2 μCi of ³³P-dATP (AMERSHAM PHARMACIA BIOTECH), using specific primers downstream and random primers upstream: AP0: TAT CGA CTC CAA G (SEQ ID No. 27); AP1: TTA GCT AGC ATG G (SEQ ID No. 28); AP2: TGC TAA GAC TAG C (SEQ ID No. 29); AP3: TTG CAG TGT GTG A (SEQ ID No. 30); AP4: TGT GAC CAT TGC A (SEQ ID No. 31); AP5: TGT CTG CTA GGT A (SEQ ID No. 32); AP6: TGC ATG GTA GTC T (SEQ ID No. 33); AP7: TGT GTT GCA CCA T (SEQ ID No. 34); AP8: TAG ACG CTA GTG T (SEQ ID No. 35); AP9: TTA GCT AGC AGA C (SEQ ID No. 36); AP10: TCA TGA TGC TAC C (SEQ ID No. 37); AP11: TAC TCC ATG ACT C (SEQ ID No. 38); AP12: TAT TAC AAC GAG G (SEQ ID No. 39); AP13: TAT TGG ATT GGT C (SEQ ID No. 40); AP14: TAT CTT TCT ACC C (SEQ ID No. 41); AP15: TAT TTT TGG CTC C (SEQ ID No. 42); AP16: TTA TCT ATA CAG G (SEQ ID No. 43); AP17: TTA TGG TAA AGG G (SEQ ID No. 44); AP18: TTA TCG GTC ATA G (SEQ ID No. 45); AP19: TTA GGT ACT AAG G (SEQ ID No. 46). The amplification parameters are 1 min at 94° C., 1 min at 40° C., 1 min at 72° C., followed by a final extension step of 5 min at 72° C. The PCR products are then separated by electrophoresis on a 6% polyacrylamide-8 M urea gel. After drying, the gel is then exposed on a Hyperfilm-HP film. The CDNA bands which are expressed differently are excised from the gel, eluted in 100 μl of sterile water, precipitated and then resuspended in 10 μl of sterile water. The cDNA fragments are then reamplified using the same pair of primers described above and subsequently cloned into the cloning vector TOPO TA PCR II (INVITROGEN, Groningen, The Netherlands).

### 4. DNA Sequencing

The cloned cDNAs are used as a matrix for the sequencing using the “PRISM Ready Reaction Dye Deoxy Terminator” sequencing kit from APPLIED BIOSYSTEMS. The samples are subjected to electrophoresis on an ABI 377 DNA sequencer; they are read automatically and then recorded using the ABI Prism software, version 2.2.1, from APPLIED BIOSYSTEMS. Homology searches in nucleotide databanks were carried out using the Basic Local Alignment Search Tool (BLAST) program.

### 5. Semiquantitative RT PCR for Detecting Gene Expression

The total cellular RNA extracted using the RNA plus kit (Q-BIOgene, Illkirch, France) is pretreated with DNAse I. Synthesis of the first strand on the polyA+ RNA was primed with a p(dT)₁₅ primer (BOERHINGER MANNHEIM, ROCHE DIAGNOSTICS, France) and carried out with the reverse transcriptase derived from the Moloney murine leukemia virus (M-MLV) (PROMEGA, Madison, Calif., USA) (1 hour at 37° C., 3 min at 92° C.). The resulting cDNA is then subjected to 25 to 35 repeat amplification cycles with the AmpliTaq Gold polymerase (APPLIED BIOSYSTEMS). PCR amplification of the GAPDH sequences is carried out in order to obtain an internal control. The specific oligonucleotide primers are as follows: GAPDH [sense strand: 5′-GCC ATC AAT GAC CCC TTC ATT GAC-3′ (SEQ ID No. 19); antisense strand: 5′-TGA CGA ACA TGG GGG CAT CAG CAG-3′ (SEQ ID No. 20)], RH116 [sense strand: 5′-GGA AGT ACA ATG AGG GCC TAC AAA-3′ (SEQ ID No. 13); antisense strand: 5′-TCC TCA GTC CTA GTA TAT TGC TCC-3′ (SEQ ID No. 14)], SSA-56 [sense strand: 5′-GAA AGA GAG GTC GCA GAG GCC TGT-3′ (SEQ ID No. 47); antisense strand: 5′-TGA TAA GGC TGA GGA AGG GAA ATG-3′ (SEQ ID No. 48)], Tat (sense strand: 5′-CTA GAC CCC TGG AAG CAT CCA-3′ (SEQ ID No. 49); antisense strand: 5′-TCG GGC CTG TCG GGT CCC CTC-3′ (SEQ ID No. 50)].

All the PCR products are then separated on 2% agarose gels and then visualized by ethidium bromide staining. Using imaging systems (IMAGE MASTER 1D PRIME, AMERSHAM PHARMACIA BIOTECH), the percentage variation in messenger RNA expression was deduced after standardization relative to the levels of the corresponding internal standards, as previously described (Amiel et al., 1999).

### 6. Cloning of the Full Length cDNA

The full length RH116 cDNA was cloned using the SMART™ RACE cDNA amplification kit (CLONTECH, Palo Alto, Calif., USA) on polyA+ RNA samples from PBMCs from a healthy patient depleted for CD8. In addition, a human spleen library (λ TriplEX™ Library CLONTECH) was screened with specific probes labeled with ³²p phosphorus according to the manufacturer's instructions.

### 7. Northern Blotting Analysis

The Northern blotting analysis was carried out using the “Multiple Tissue Northern” (MTN™) kit (CLONTECH) according to the manufacturer's instructions. The polyA+ RNA was hybridized with the oligonucleotides 5′-CGT GCT GAT TCC TCA GTC CTA GTA TAT TGC-3′ (SEQ ID No. 26) and 5′-GCA TCT GCA ATG GCA AAC TTC TTG CAT GGC-3′ (SEQ ID No. 18) derived from the RH116 cDNA sequence and labeled at the end with ³²P phosphorus. The membranes were rehybridized with a human β-actin CDNA probe.

### 8. Expression of the Protein Labeled with a His-Tag and Production of Mouse Polyclonal Antibodies

A partial fragment corresponding to the first 335 amino acids of the RH116 messenger RNA was amplified using specific oligonucleotides (SEQ ID No. 21 and SEQ ID No. 22) and was subcloned into the vector pQE-81 (QIAGEN, Courtaboeuf, France), cleaved beforehand with the BamHI and SalI enzymes. The resulting plasmid, pQE-81-His6-RH116₁₋₃₃₅, is then transformed into E. coli TOP10F′ cells. The transformed cells are then cultured and induced with 1 mM of isopropyl-1-thio-β-D-galactopyranoside for 5 hours. Purification under denaturing conditions is carried out using Ni-NTA beads according to the manufacturer's recommendations (QIAexpressionist™ QIAGENE).

The RH116₁₋₃₃₅ partial recombinant protein (50 μg), emulsified in complete Freund's adjuvant (SIGMA, Saint-Louis, Mich., USA), is used to immunize a mouse by intraperitoneal injection. After 30 days, the animals are stimulated with 50 μg of the same recombinant protein emulsified with incomplete Freund's adjuvant (SIGMA) and 15 days later with 50 μg of the same protein without adjuvant. The sera are collected 10 days after the final stimulation and are tested for the level of anti-RH116 antibodies. The immunoglobulins are then purified from the antisera according to the method using caprylic acid (Steinbuch and Audran, 1969), and are concentrated by precipitation with saturated ammonium sulfate. Before the immunization, the sera are collected and used as a negative control. The antisera and immunoglobulin activities are tested by ELISA, as described below.

### 9. Analyses by Indirect Immunofluorescence

For the analyses by indirect immunofluorescence, the HeLa-CD4+ cells are cultured on chamber slides (NALGE, Nunc, Rochester, N.Y., USA) and fixed and permeabilized with a methanol/acetone (2V/1V) mixture for 10 min at −20° C. After having been humidified in PBS containing 1% of bovine serum albumin (BSA) for 30 min, the samples are incubated with the first antibody at ambient temperature for 45 min in PBS containing 0.5% BSA. The normal mouse serum and the antiserum against RH116₁₋₃₃₅ is used at a dilution of 1/20; the antihistone 1 monoclonal IgG2a antibody (SANTA CRUZ BIOTECHNOLOGIES, USA) and a control antibody, which corresponds to this type of isotype, are used at a concentration of 1/50. After 3 washes in PBS, the samples are stained with an anti-mouse goat IgG antibody conjugated to FITC (fluorescein isothiocyanate) (SIGMA). After several washes, the samples are incubated for 3 min in PBS containing 0.5 μg/ml of propidium iodide, and then examined by fluorescence microscopy.

### 10. Subcellular Fractionation

The subcellular fractionation is prepared as described in Li—Ru et al. (1999). Briefly, to prepare the total cell lysates, the cells are lysed in a buffer containing 10 mM Tris-HCl, pH 7.1, 1 mM EDTA, 1% triton X-100, 1 mM PMSF, 1 mM Na₃VO₄, 10 μM E-64 (trans-epoxy-succinyl-L-leucylamido-(4-guanidino)-butane), 1 μg/ml of pepstatin and 0.1% of aprotinin. To prepare the nuclear fractions, the cell pellets are incubated in a hypotonic buffer (20 mM HEPES, pH 7.4, 1 mM MgCl₂, 10 mM KCl, 0.5% Nonidet P40, 0.5 mM dithiothreitol (DTT), 1 mM PMSF, 1 mM Na₃VO₄, 10 μM E64, 1 μg/ml pepstatin and 0.1% aprotinin) at 4° C. for 30 min. After centrifugation, the resulting pellets containing the nuclei are resuspended in a high ionic strength buffer (1 mM HEPES, pH 7.4, 20% glycerol, 0.4 M NaCl, 1 mM MgCl₂, 10 mM KCl, 0.5 mM DTT, 1 mM PMSF, 1 mM Na₃VO₄, 10 μM E64, 1 μg/ml pepstatin, 10 μg/ml leupeptin and 0.1% aprotinin. The quality of the fractionation is controlled by Western blotting using monoclonal antibodies against histone 1 (SANTA CRUZ BIOTECHNOLOGY) (nuclear protein) and against the 58K Golgi protein (SIGMA) (cytoplasmic protein).

### 11. Analysis by Western Blotting

The cell lysates are mixed with a 2×SDS loading buffer, incubated at 100° C. for 5 min, then separated by electrophoresis on a 15% polyacrylamide SDS-PAGE gel and, finally, transferred onto a nitrocellulose membrane (Hybond-C, Amersham). The membranes are saturated with PBS (phosphate buffered saline) containing 5% of fat-free milk for 1 hour at ambient temperature, followed by incubation of the primary antibody (1/100 dilution for the anti-histone 1 antibody; 1/1 000 dilution for the anti-58 K Golgi antibody) at 4° C. overnight. After washes with PBS-0.1% Tween-20, the membranes are incubated with a second antibody conjugated to horseradish peroxidase (1/1 000) (SIGMA) at ambient temperature for 1 hour. The bands transferred onto the membranes are detected by incubation with an enhanced chemiluminescence (ECL) reagent (Amersham) and exposed to a KODAK XOMAT film.

### 12. Immunoprecipitation

500 μg of each cell extract are radiolabeled with 500 μCi of ¹²⁵I iodine, using the chloramine T method. 3×10⁶ CPM of cell extracts are pretreated for 2 hours with a normal mouse serum and 50 μl of protein G sepharose (PHARMACIA) in a TNSTEN buffer (50 mM Tris-HCl, pH 8.2, 0.5 M NaCl, 0.1% SDS, 0.5% Triton X100, 5 mM EDTA, 0.02% NaN₃, with 0.1% of aprotinin). The immunoprecipitations are carried out by adding 20 μg of immunoglobulins purified from the anti-RH116₁₋₃₃₅ serum or from normal serum and 50 μl of protein G sepharose. After incubation overnight at 4° C., the sepharose beads are washed 10 times with a TNSTEN buffer and then resuspended then boiled in a 2×-concentrated SDS-PAGE loading buffer. The proteins are separated on an SDS-PAGE gel at 8%, and then analyzed by autoradiography.

### 13. DNA Transfection

All the plasmids used are prepared using endotoxin-free materials (“EndoFree™”, Giga Kit, QIAGEN). For the transient expression in HeLa-CD4+ cells, an XhoI-BamHI fragment comprising the RH116 cDNA is subcloned into a mammalian expression vector pEGFP-N1 (CLONTECH). The resulting chimeric construct consists of a full length RH116 protein fused to the carboxy-terminal end of the green fluorescent protein (GFP). Fusion in a correct reading frame for the cDNA is controlled by sequencing. The coding sequence of any protein such as SSA (available in the inventors' laboratory) was subcloned into the vector pEGFP-N1 (GFP-SSA) and used as a control. For the positive control, the inventors studied the effects of transient expression of Tat cloned into pCR3, as previously described (Billaut-Mulot et al., 2001); the native pCR3 constitutes the corresponding control. The transfection is carried out using Effectene (QIAGEN) as transfecting reagent and used according to the manufacturer's protocol. Briefly, the day before transfection, 2×10⁵ cells are seeded per well in 12-well plates and are subsequently transfected using 1 μg of DNA and 10 μl of Effectene in experiments carried out in triplicate. The cells are infected with HIV-1_{LAI} 24 hours after transfection.

### 14. In Vitro Infections

The T-cell strain with a tropism for HIV-1_{LAI} is obtained from the Laboratoire de Virologie Centrale de Lille [Central Virology Laboratory of Lille], France. To infect transfected cells, 5×10⁴ CPM of viral reverse transcriptase (RT) are added to each well. The cells are incubated overnight at 37° C. The viruses are then removed and the cells are washed twice and a fresh medium is added. One day after infection, and for the next 4 days, the supernatants are collected from each of the wells and the cells are recovered and counted. Viral replication is evaluated by detecting the HIV-1 DNA and RNA 24 hours after infection or by detecting the presence of the p24 protein in the supernatants between 2 days and 5 days after infection.

### 15. Detection of HIV-1 DNA and RNA

The total cellular DNA is extracted from the cells infected with HIV-1, 24 hours after infection, and then subjected to 35 amplification cycles with the “AmpliTaq Gold” DNA polymerase. The PCR amplification of the β-actin sequences is carried out with the oligonucleotides 5′-GGG TCA GAA GGA TTC CTA TG-3′ (SEQ ID No. 51) and 5′-GGT CTC AAA CAT GAT CTG GG-3′ (SEQ ID No. 52) in order to standardize the equivalence between the cells. The HIV-1 proviral DNA of each sample is measured using the primer GAG06 (5′-GCI TTI AGC CCI GAA GTI ATA CCC ATG-3′; SEQ ID No. 53) and GAG04 (5′-CAT ICT ATT TGT TCI TGA AGG GTA CTA G-3′; SEQ ID No. 54). To measure the level of HIV-1 RNA, the total cellular RNA is extracted using the RNAplus kit (Q-BIOgene) and then amplified using rTth polymerase (Applied Biosystems) in the presence of the pair of primers GAG06-GAG04 to detect the unspliced HIV-1 GAG-POL mRNA, and in the presence of the pair of primers BSS (5′-GGC TTG CTG AIG NGC ICA CIG CAA GAG G-3′; SEQ ID No. 55)-KPNA (5′-AGA GTI GTG GTT GNT TCN TTC CAC ACA G-3′; SEQ ID No. 56) to detect the intermediate size of the single-spliced messenger RNA as previously described (Amiel et al., 1999). All the PCR products are separated on an acrylamide gel and visualized by ethidium bromide staining. Using imaging systems (Image Master 1D Prime; Amersham PHARMACIA BIOTECH), the change in expression of the HIV-1 DNA and of the RNA is deduced after standardization relative to the corresponding internal standard controls (respectively GAPDH or β-actin). Briefly, the HIV RNA levels are standardized to the β-actin level by calculating the ratio of the volume of the HIV RNA band to that of β-actin. Thus, the amount of increase in the expression of HIV is deduced by comparing the cells transfected with RH116, SSA or Tat, with the HeLa CD4+ cells transfected with GFP or pCR3.

### 16. p24 Assay

The viral replication is evaluated by measuring the p24 antigen level in the culture supernatants using the HIV-1 p24 antigen assay kit (Coulter, Miami, USA) according to the manufacturer's instructions.

### 17. Detection by ELISA of Autoantibodies in the Sera of HIV+ Patients

To evaluate the presence of antibodies against RH116 in the sera of HIV-1 patients, a group of 32 individuals infected with HIV-1 were tested before and after treatment with potential antiretroviral agents. The group consisted of 8 women and 24 men with an average age of 37 (range 25-64). Before the start of the antiretroviral therapy, the mean viral load in the plasma was 421 326 copies/ml and the mean number (+standard deviation) of CD4+ cells was 150 (+14) cells/μl. After an average duration of treatment of 14 months with two reverse transcriptase inhibitors and a protease inhibitor, the viral load fell to a mean level of 3 272 copies/ml and the number of CD4+ cells increased to 313 (+23) cells/μl. The antibody levels in the serum of the patients are compared with those present in the serum of 40 healthy controls of corresponding sex and age. The presence of auto-antibodies against RH116 in the sera was sought by ELISA. Briefly, the plates are coated with 1 μg/ml of RH116₁₋₃₃₅ in a coating buffer (30 mM Na₂CO₃, 70 mM NaHCO₃) for 3 hours a 4° C. After 3 washes in PBS containing 0.05% of Tween-20 (PBS-Tween), the plates are incubated for 2 hours with sera diluted from 1/300 to 1/2 700. The plates are then washed and incubated overnight at 4° C. with a human IgG antibody (SIGMA) conjugated to peroxidase. After 3 washes with PBS-Tween and one wash with PBS, the plates are developed for 30 minutes using 0.5 mg/ml of an O-phenylenediamine dihydrochloride substrate (SIGMA) and 0.1% H₂O₂, and the reaction is stopped by adding 50 μl/well of 1N HCl. The absorbence values are read at 492 nm using an automatic microplate reader (TITERTEK MULTISKAN, LABYSYSTEMS, Finland). Similar protocols are used to evaluate the presence of anti-RH116 antibodies in the sera of immunized mice, with the exception that the conjugate used is an anti-mouse antibody in place of an anti-human IgG antibody.

### Example 1

### Identification of the RH116 Gene Modulated by Murabutide, by DD-RT-PCR

Samples of RNA from nonstimulated PBMCs and from CD8-depleted PBMCs stimulated with murabutide and obtained from an HIV-1 patient are analyzed by DD-RT-PCR. The inventors selected more than 130 cDNA fragments differentially expressed after treatment of PBMCs of an HIV+ patient with murabutide. These fragments were subcloned into the vector Pcr2.1 (Invitrogen), and then sequenced by automatic sequencing (ABI Prism 377, Perkin-Elmer). The sequences were analyzed for homology searches using the databanks and the Basic Local Alignment Search Tool (Blast 2) server of the NCBI.

The expression of a certain number of genes appears to be positively or negatively regulated after treatment with murabutide for 6 or 24 hours. Among these, 3 genes correspond to alu repeats, 20 sequences correspond to ESTs or genomic sequences of the databanks, and 49 sequences are well-characterized genes. Among the genes regulated by murabutide, 28 exhibit an increased expression and 21 have their expression inhibited. The expression profile of 14 genes positively regulated by murabutide and of 7 genes negatively regulated by murabutide was confirmed by RT-PCR or by reverse Northern blotting in the form of a “dot-blot” (data not shown) on RNA extracts of CD8-depleted PBMCs obtained from 3 other patients. The modulation of expression by murabutide was also verified among the sequences identified by DD-RT-PCR which correspond to ESTs or the genomic sequences of the databanks. Thus, 6 clones exhibit an increased expression: 4 clones (clones 1, 71, 87 and 99) subsequent to treatment for 6 hours, 2 clones (clones 11 and 89) subsequent to treatment for 24 hours. Furthermore, 4 sequences identified induce inhibition of expression: 2 clones (clones 7 and 10) subsequent to exposure to murabutide for 6 hours, and 2 clones (clones 62 and 96) subsequent to exposure to murabutide for 24 hours.

The expression profile of the new gene regulated by murabutide and corresponding to clone 10 was studied by semi-quantitative RT PCR and the corresponding full length cDNA was cloned and characterized.

### Example 2

### Full Length Cloning of the RH116 Gene Modulated by Murabutide

The full length DNA sequence corresponding to clone 10 was obtained with a 5′ and 3′ RACE strategy. Using the cDNA fragments obtained by DDRT PCR as a basis, a first cycle of 5′ RACE and of 3′ RACE made it possible for the inventors to characterize the polyA+ end of this new gene and to extend the cDNA sequence in the 5′ portion of the gene. The complete 5′ end of the cDNA was obtained after 3 steps of 5′ RACE. Screening a cDNA library made it possible for the inventors to confirm the full length cDNA sequence. The complete nucleotide sequence was obtained by determining the overlapping sequences of 2 different clones. The full length cDNA sequence was submitted to GeneBank (accession No. AY 017378).

More precisely, from a fragment 164 bp long (SEQ ID No. 3) obtained by DD-RT-PCR, the inventors synthesized two specific primers, including F1: 5′ TGA TGA GGG TGG TGA TGA TGA GTA TTG TG 3′ (SEQ ID No. 4), in order to perform a first amplification by 5′ and 3′ RACE. The inventors were thus able to obtain a 1 284 bp fragment by virtue of the amplification using F1. This fragment was sequenced in several steps using the specific internal primers F2 (5′-GCA GTG AGT TCA AAC CCA TGA CAC AGA ATG-3′) (SEQ ID No. 5) and R2: (5′-CAG CAT TCT GAA TAG TCA AGA TTG GGA AAT G-3′) (SEQ ID No. 6); the sequence of this fragment corresponds to the sequence SEQ ID No. 7. This has an open reading frame of 380 amino acids up to a potential stop codon which is followed, after 119 bp, by a polyA+ tail (FIG. 1).

In order to obtain the 5′ portion of the cDNA, the inventors synthesized a primer, R3 (SEQ ID No. 8), which corresponds to the sequence complementary to F1; the primer R3 made it possible, after PCR, to obtain a 194 bp sequence (symbolized with a dotted line . . . in FIG. 2A).

Based on this sequence, the inventors synthesized a new primer, R8 (SEQ ID No. 9): 5′-GTA GGG CCT CAT TGT ACT TCC TCA AAT-3′), in order to determine the sequence in the position 5′ of the cDNA; a PCR reaction made it possible to obtain a fragment of approximately 1 200 bp (symbolized by a dashed line - - - in FIG. 2a) (SEQ ID No. 10). Complete sequencing of the fragment was carried out in several steps using internal oligonucleotides R8-seq 1 (5′ CTC CAA CAC CAG GTG AAG CTG 3′) (SEQ ID No. 11) and R8-seq 2 (5′ CAG ATG AAG AGA ATG TGG CAG 3′) (SEQ ID No. 12). The reading frame remains open and makes it possible to identify a reading frame of 853aa.

Using these two sequences, the inventors synthesized two primers, HELI 1 (5′-GGA AGT ACA ATG AGG GCC TAC AAA-3′) (SEQ ID No. 13) and HELI 2 (5′-tcc tca gTc cta gta tat tgc tcc-3′) (SEQ ID No. 14), taken on the F1 and R3 fragment, respectively, in order to carry out RT-PCRs to analyze the differential expression of the corresponding mRNA (see example No. 4).

Using the sequence SEQ ID No. 10, the inventors synthesized a new primer, R16 (5′-CTA AGC AGC TGA CAC TTC CTT CTG CCA AAC TTG TGT CTG-3′) (SEQ ID No. 15), in order to extend the cDNA back in the 5′ direction; a PCR reaction made it possible to obtain a 964 bp fragment (symbolized by a broken line ( - - - ) FIG. 2b).

The 964 bp 5′ end obtained after the PCR reaction (5′RACE) contains a potential ATG codon which is preceded by a STOP codon, the presence of which will be subsequently confirmed. This strategy therefore made it possible for the inventors to obtain an open reading frame (ORF) of 1 025 amino acids.

The complete sequence of the cDNA corresponding to RH116 is now 3 372 bp and corresponds to SEQ ID No. 1 and encodes a 1 025 aa protein which corresponds to SEQ ID No. 2.

A PCR strategy using two primers, F4: (5′-ggg ccc tgt gga caa cct cgt cat tgt-3′) (SEQ ID No. 16) and R14: (5′-CCA GAG TGG CTG TTT ACA TTG CCA AGG ATC ACT-3′) (SEQ ID No. 17), specific for the sequence SEQ ID No. 1, was developed in order to confirm the presence of the ATG translation initiation codon and also the presence of the STOP codon (TGA) upstream of this. For this, the inventors carried out a PCR on a 5′ RACE matrix using the two primers mentioned above, and obtained a fragment of expected size, which was cloned and sequenced. The sequence of this fragment confirmed the presence of the TGA codon before the initiating ATG codon, thus confirming the inventors have the complete copy of the cDNA.

The cDNA sequence (3 372 base pairs) contains an initiation codon at position 155 and an open reading frame (ORF) of 3 075 base pairs, and also a 3′ nontranscribed region of 141 base pairs with a consensus polyadenylation signal AATAAAA located 24 base pairs upstream of a polyA+ tail of 21 base pairs. The ORF encodes a 1 025 amino acid polypeptide (FIG. 2B) with a calculated molecular mass of 116 kdaltons and an isoelectric point of 5.2.

### Example 3

### Study of Comparison of the Full Length RH116 Sequence with the Sequences of the Databases

The inventors compared the deduced amino acid sequence of 1 025 amino acids with the sequences present in the databases.

The inventors identified, in this sequence, conserved domains belonging to the RNA helicase superfamily and, more precisely, the family of proteins known to have ATP-dependent RNA helicase functions. Among the helicase classes based on this sequence homology (Luking et al., 1998) are the proteins called “DEAD-BOX”. An important characteristic of all “DEAD-BOX” RNA helicases is the presence of characteristic motifs separated from one another by a conserved distance (Luking et al., 1998; Linder and Daugeron, 2000). Despite minor differences, these motifs are present in the new cloned sequence (FIG. 3A and 3B). As a result, the inventors consider that this protein, called RH116 (for RNA helicase 116 kdaltons), constitutes a member of the “DEAD-BOX” protein subfamily of ATP-dependent RNA helicases. The inventors also noted the presence of a KKKK motif at amino acid position 349, but no bipartite nuclear localization signal motif A could be clearly identified. Eight N-glycosylation sites were detected and potential phosphorylation sites were found for cAMP-dependent kinase and also protein kinase C; the biological significance of these sites has not been studied.

In the course of the searches carried out in the databanks, the inventors noted that the RH116 protein exhibited, besides its general sequence homology with the members of the “DEAD-BOX” protein family, a strong % homology with a hypothetical human RNA helicase until now not characterized, called “RIG-1” (FIG. 3b C), described by Y. W Sun (Genbank, accession number: AF038963). The sequence alignment is given in FIG. 3b C. The total amino acid sequence identity between the two proteins is 31% and the similarity which includes conservative exchanges is 44%.

Searching various databanks made it possible for the inventors to find parts of the nucleotide sequence in other different genomic clones. One part of the sequence is found in the NH0576116 clone registered under the accession number gb/AC007750, the second part is found in the genomic clone RP11-214A4 registered under the No. AC108176. Mention should also be made of the ESTs (expressed sequence tags) registered under the accession numbers AW 589567, AW 152541 and AW 189584 in the EMBL databank, which exhibit, respectively, 100%, 99% and 97% homology with, respectively, the fragments of sequence 2879-3350, 2870-3350 and 2818-3350 of the sequence SEQ ID No. 1.

### Example 4

### Study by Northern Blotting of the Expression of the RH116 mRNA in Various Tissues

PolyA+ RNAs of various tissues were tested by Northern blotting using oligonucleotides labeled at their end and derived from the RH116 cDNA sequence; the nucleotide sequence 5′-GCA TCT GCA ATG GCA AAC TTC TTG CAT GGC-3′ (SEQ ID No. 18) and the nucleotide sequence 5′-CGT GCT GAT TCC TCA GTC CTA GTA TAT TGC-3′ (SEQ ID No. 26), specific for the coding sequence, was synthesized and labeled with ³²P (T4 Polynucleotide Kinase, Amersham) in order to serve as a probe to perform Northern blotting. Hybridization of a membrane containing 2 μg of polyA+ RNA (Clontech) revealed the result given in FIG. 4.

A specific signal corresponding to a messenger RNA with an estimated size of approximately 3.5 kb was detected, indicating that the cDNA sequence isolated was a full length sequence (FIG. 4). The level of expression of the messenger RNA in 12 human tissues (brain, heart, skeletal muscle, colon, thymus, spleen, kidney, liver, small intestine, placenta, lung and peripheral blood leukocytes) was studied. The strength of the radioactive signal is slightly reduced in the brain, colon, thymus, small intestine and peripheral blood leukocytes, and a very strong radioactive signal is present in the heart and kidney.

### Example 5

### Study by RT PCR of the Differential Expression of RH116 in the PBMCs of HIV+ Patients and of Healthy Controls After Treatment, or Without Treatment, with Murabutide

Expression of the RH116 messenger RNA is inhibited by murabutide. The inventors also studied whether the RH116 messenger RNA was a new gene modulated by murabutide, using semiquantitative RT PCR using specific oligonucleotides derived from the RH116 cDNA.

PBMCs of patients (P) infected with HIV or of control healthy donors (C) are isolated, depleted of CD8+ lymphocytes (Dynabeads, Dynal) and stimulated with phytohemagglutinin, PHA (5 μg/ml), for 3 days. The cells are then treated or not treated with murabutide (10 μg/ml) in the presence of interleukin 2 (IL2) (10 U/ml) in an RPMI medium supplemented with 10% of fetal calf serum (SVF) for 6 hours or 24 hours in a proportion of a minimum of 5×10⁶ cells per condition. After treatment, the RNA of the cells is extracted (RNAplus, Quantum-bioprobe), then treated with DNase (Boerhinger) and reverse transcribed (RT) using an oligo(dT) in the presence of the Mu-MLV reverse transcriptase (Superscript II, Gibco). The quality of the RTs is verified by PCR (25 cycles) using primers specific for GAPDH (5′GCC ATC AAT GAC CCC TTC ATT GAC 3′) (SEQ ID No. 19) and (5′ TGA CGA ACA TGG GGG CAT CAG CAG 3′) (SEQ ID No. 20) on 20, 100 and 500 ng of total RNA. The inventors then carried out RT-PCRs (35 cycles) using primers, Heli 1 and Heli 2, specific for the new RH116 polypeptide (5′ GGA AGT ACA ATG AGG GCC TAC AAA 3′) (SEQ ID No. 13) and (5′ TCC TCA GTC CTA GTA TAT TGC TCC 3′) (SEQ ID No. 14). The number of amplification cycles was determined beforehand (35 cycles). The amplified fragments are visualized on an agarose gel (1%) in the presence of ethidium bromide, and then quantified using the Imager master program (Pharmacia). For each dilution, the value given for the gene studied is related to that of GAPDH (Ratio=R). For each patient and each time (6 h or 24 h), the R of the cells treated with murabutide is related to that of the untreated cells. The results are then expressed as % increase or % inhibition of expression of the gene compared with the untreated cells. It should be noted that, for each dilution tested, the R can vary slightly; the mean of the Rs was produced by taking care to always be in the linear phase of amplification.

This study was carried out on 12 patients and 10 healthy controls. The study on the patients shows a significant inhibition of expression of the RH116 gene after treatment for 6 or 24 h with murabutide, compared with the untreated cells. In addition, the study carried out on PBMCs of healthy donors reveals a very significant increase in expression of the RH116 gene, in particular after 6 hours of treatment with murabutide.

FIG. 5 represents the RT-PCR results obtained on the PBMCs of patients after 6 and 24 hours of treatment.

A representative RT PCR carried out on CD8-depleted PBMCs obtained from patients infected with HIV-1 is given in FIG. 5A, and shows a significant inhibition of expression of the messenger RNA subsequent to treatment for 6 or 24 hours with murabutide.

The results given in FIG. 5B give a mean percentage inhibition of expression of the RH116 messenger RNA and demonstrate that the treatment, with murabutide, of the CD8-depleted PBMCs of 5 patients induces a considerable inhibition of expression of the RH116 messenger RNA with a maximum mean inhibition (90%) observed in patient No. 5 after 6 hours of treatment with murabutide, and this effect can be maintained after 24 hours, the maximum mean inhibition (57%) being observed in patient No. 3.

### Example 6

### Expression of the Recombinant Protein in an E. coli Bacterial System (pQE)

A PCR was carried out on spleen cDNA using nucleotide primers corresponding to the ATG: Heli ATG: (5′-TGA GAG GAT CCG ATG TCG AAT GGG TAT TCC 3′) (SEQ ID No. 21) and to the STOP (5′-AAT GTC GAC CTA ATC CTC ATC ACT AAA TAA-3′) (SEQ ID No. 22).

The fragment was cloned into the vector pQE80 (Qiagen) digested with the BamHI/Sal I restriction enzymes.

After transformation of TOP 10F′ bacteria, the expression of the recombinant protein is induced with IPTG; the expression time was optimized and estimated to be two hours; indeed, after 5 hours of induction, the inventors do not detect any recombinant protein. The protein is weakly expressed (approximately 500 μg per 500 ml of culture) and is in soluble form.

### Example 7

### Expression of the Recombinant Protein in a Eukaryotic System

Since the “RH116” protein is expressed in the cytoplasm or in the nucleus of mammalian cells, the inventors developed a strategy of overexpression of the recombinant protein in eukaryotic cells in order to assess its role in regulating HIV.

For this, the complete copy of the cDNA was amplified using the primers heli-GFP-ATG (Xho I) (5′-TGA GAG CTC GAG ATG TCG AAT GGG TAT TCC ACA GAC-3′) (SEQ ID No. 23) and Heli-GFP (Bam HI) (5′-TGT TTA TTT AGT GAT GAG GAT CGG GAT CCG ATT GAA-3′) (SEQ ID No. 24); the amplified fragment is cloned into the vector pEGFP digested with Xho I/Bam HI, and then sequenced. The cDNA encoding green fluorescent protein (GFP) is located 3′ of the cloned insert.

In addition, the complete copy of the CDNA was amplified using the primers Heli ATG Bam (5′-TGA GAG GAT CCG ATG TCG AAT GGG TAT TCC-3′) (SEQ ID No. 21) and Heli STOP XhoI (5′-ttc aat ctc gag atc ctc atc act aaa taa aga-3′) (SEQ ID No. 25); the amplified fragment is cloned into the vector pcDNA6 digested with Bam HI/Xho I. In this system, the protein is fused to 6 histidines and to a protein V5 against which monoclonal antibodies are available.

### Example 8

### Transfection in Eukaryotic Cells

Transfection experiments were carried out in cos-7 cells and Hela cells using the recombinant plasmids pEGFP and pcDNA6 containing the complete sequence of the RH116 polypeptide as described in example 5.

The transfections were carried out (Effectene-Qiagen) with 1 μg of recombinant plasmid and 10 μl of Effectene. An RT-PCR analysis made it possible to conform the overexpression of the mRNA encoding the RH116 polypeptide. Expression of the fused recombinant proteins was verified by flow cytometry (fusion with GFP) or by Western blotting (fusion with V5-HIS6).

### Example 9

### Molecular Characterization of the cDNA Encoding the RH116 Protein

The molecular characterization of the native RH116 protein was carried out using immunoglobulins purified by mouse serum against the partial recombinant RH116₁₋₃₃₅ protein. Cell labeling and immunoprecipitation generate a protein product of 130 to 140 kdaltons in agreement with the calculated molecular mass of the encoded protein, which must be highly glycosylated. The RH116 protein is immunoprecipitated from total cell lysates obtained from U937 cells and from fractions enriched in nuclear extracts (FIG. 6A). Similar results are obtained on HeLa cells (data not shown). FIG. 6B presents the subcellular fractionation quality control and shows enriched nuclear extracts which exhibit an increased expression of histone 1 (34 kdaltons) only in the total extract and less expression of cytoplasmic proteins (60 kdaltons and 40 kdaltons approximately).

### Example 10

### Study of the Immunolocalization of the RH116 Protein

In order to examine the cellular location of the native RH116 protein, the inventors carried out immunocytochemistry experiments on monolayer cultures of HeLa-CD4+ cells (FIG. 7). A mouse antibody against the RH116₁₋₃₃₅ protein strongly stains the cytoplasm of HeLa-CD4+ cells (FIG. 7C). A normal mouse serum is used as negative control (FIG. 7A) and nuclear staining is observed using an anti-histone 1 monoclonal antibody (FIG. 7B). The localization of the RH116 protein is also confirmed by studies of immunolocalization in U937 cells (data not published). In addition, the intracellular localization of the RH116 protein in HeLa-CD4+ cells transfected with a chimeric cDNA construct encoding the full length RH116 protein fused to a carboxy-terminal end of GFP (GFP-RH116) is different. Specifically, the labeling of the GFP-RH116 protein expressed in the transfected HeLa-CD4+ cells shows a cytoplasmic and nuclear localization (data not shown). The latter observation is more solid than the immunoprecipitation analyses and the putative activity of members of the “DEAD-BOX” protein subfamily of RNA helicases.

### Example 11

### RH116 Increases Expression of the P24 Viral Protein

With the aim of elucidating the putative role of the RH116 protein, the expression of which is inhibited by the “HIV inhibitor” activity of murabutide, the inventors sought to determine whether RH116 could play a role in regulating the expression of the virus. Thus, the inventors began a study of the expression of P24 by HeLa-CD4+ cells transfected with the GFP-RH116 protein and infected 24 hours later with the HIV-_{LAI} virus.

Microscopic examination of a culture at various times and analysis by trypan blue staining revealed that infection of cells transfected with GFP-RH116 affects the viability of the cells compared to that of cells transfected with GFP or of GFP-SSA (an unrelated protein) cells. Thus, the inventors standardized the levels of P24 antigens per 5×10⁵ cells. The study of the expression of the P24 viral protein in the supernatants of HeLa-CD4+ cells expressing GFP, GFP-RH116 or GFP-SSA was carried out over 5 independent experiments. The study of the expression of the P24 viral protein in the supernatants of HeLa-Cd4+ cells transformed with pCR3 and with pCR3-Tat was carried out in 3 independent experiments. FIGS. 8A and 8B give results obtained. FIG. 8A shows that the expression of the viral P24 antigen (in nanograms/ml±the standard deviation) in the supernatants of HeLa-CD4+ cells transfected with GFP-RH116 is dramatically inhibited at day 3 (12.1±1.48 ng/ml) up to day 5 (9 770±648 ng/ml) compared with the cells expressing GFP (2.65±0.5 ng/ml and 690±272 ng/ml, respectively, at days 3 and 5).

The level of P24 antigen in the supernatant of the cells transfected with pCR3-Tat is given in FIG. 8B (in nanograms/ml+the standard deviation). As expected, the results show a considerable increase in the release of P24 by the cells transfected with Tat from day 2 (2.2±0.18 ng/ml) to day 5 (2.994±391 ng/ml) compared with the cells transfected with pCR3 at day 2 (1±0.08 ng/ml) and at day [lacuna] (74±22 ng/ml). FIG. 8C gives the fold increase obtained in 3 or 5 independent experiments. These results clearly indicate that overexpression of GFP-RH116 induces a considerable increase in P24 from day 2 (3.43±0.17) to day 5 (18.2±10) compared with the overexpression of GFP-SSA (day 2: 1.18±0.25; day 5: 1.35±0.37), and an equivalent increase is obtained with overexpression of TAT (day 2: 3.0±8.42; day 5: 18.7±4.2).

### Example 12

### The RH116 Protein Regulates the Expression of Viral P24 mRNA

To determine the mechanism of the increase in expression of P24 in response to overexpression of RH116, the inventors isolated the total RNA of transfected and infected HeLa-CD4+ cells and then determined the level of unspliced, single-spliced or intermediate-size messenger RNA by semiquantitative RT PCR. The fold increase in HIV messenger RNA expression is obtained in 3 separate experiments (mean+standard deviation) (data not given). First of all, the cells transfected with GFP-RH116 have a higher level of unspliced RNA than the cells transfected with GFP-SSA compared with the cells transfected with GFP alone (respectively 3.14+1.5 and 0.81+0.12). The intermediate-sized or single-spliced HIV-1 mRNAs are also increased in the cells transfected with GFP-RH116 (2.81+0.73) compared with the cells transfected with GFP-SSA (0.91+0.14).

As regards the overexpression of Tat, results are obtained from two independent experiments; the level of unspliced and intermediate-sized or single-spliced messenger RNA is, respectively, 4.75±1.25 and 4.18±0.31. An RT PCR analysis obtained 3 days after infection is shown in FIG. 9. FIG. 9A indicates that the overexpression of the RH116, SSA or TAT mRNAs is in the transiently transfected cells. FIG. 9B illustrates the increase in the unspliced, or intermediate-size or single-spliced mRNAs of the over-expressed RH116 (on the right) and TAT (on the left) proteins. The study of the formation of the proviral DNA (FIG. 8C) reveals that there is no significant difference in any of the transfected cells. These results suggest that the regulation of expression of HIV by RH116 occurs at the transcriptional or post-transcriptional level.

### Example 13

### The RH116 Protein Constitutes an Auto-Antigen in Patients Infected with HIV-1

The question of whether RH116 may be an auto-antigen was studied by ELISA analysis using sera obtained from healthy donors and those obtained from HIV-1 patients tested before and after antiretroviral treatment. The results given in FIG. 10 significantly demonstrate an increase in the autoantibody levels in the sera from patients compared with those of healthy controls. In addition, the high levels of RH116 auto-antibodies observed in the untreated HIV-1 patients are always significant even after significant treatment with effective antiretroviral agents.

### References

Amiel et al. (1999) J. Infect Dis. 179:83-91.

Billaut-Mulot et al. (2001) Vaccine In press.

Blundell (1996) Nature 384: 23.

Buckholz (1993) Curr. Op. Biotechnology 4:538.

Carter (1993) Curr. Op. Biotechnology 3:533.

Duck et al. (1990) Biotechniques 9:142.

Edwards and Aruffo (1993) Curr. Op. Biotechnology 4:558.

Epstein (1992) Medecine/Sciences 8:902.

Fruehleis et al. (1987) Int. Ed. Engl. 26:403.

Godbout et al. (1998) J. Biol. Chem. 273:21161.

Gorbalenya et al. (1988) Nature 333:22.

Guatelli et al. (1990) Proc. Natl. Acad. Sci. USA 87:1874.

Jaramillo et al. (1991) Mol. Cell. Biol. 11:5992.

Kievitis et al. (1991) J. Virol. Methods 35:273.

Köhler and Milstein (1975) Nature 256:495.

Kwoh et al. (1989) Proc. Natl. Acad. Sci. USA 86:1173.

Lain et al. (1990) Nucleic Acid. Res. 18:7003-7006.

Landegren et al. (1988) Science 241:1077.

Lee and Hurwitz (1993) Drosphila J. Biol. Chem. 268:16822-16830.

Liang and Pardee (1992) Science 257:967-971.

Linder et al. (1989) Nature 337:121-122.

Linder and Daugeron (2000) Nat. Struct. Biol. 7:97-99.

Li-Ru et al. (1999) J. Virol. 73:2841-2853.

Luckow (1993) Curr. Op. Biotechnology 4:564.

Luking et al. (1998) Crit. Rev. Biochem. Mol. Biol. 33:259-296.

Matthews et al. (1988) Anal. Biochem. 169:1-25.

Miele et al. (1983) J. Mol. Biol. 171:281.

Neddleman and Wunsch (1970) J. Mol. Biol. 48:443.

Olins and Lee (1993) Curr. Op. Biotechnology 4:520.

Pause and Sonenbery (1992) EMBO J. 11:2643-2654.

Pearson and Lipman (1988) Proc. Natl. Acad. Sci. USA 85:2444.

Perricaudet et al. (1992) La Recherche 23:471.

Perun et al. (1989) Eds Computer-aided drug design (Marcel Dekker Inc. New York).

Rohlmann et al. (1996) Nature Biotech. 14: 562.

Rolfs, A. et al. (1991) Berlin: Springer-Verlag.

Rozen et al. (1990) Mol. Cell. Biol. 10:1134.

Segev (1992) Kessler C. Springer Verlag, Berlin, N.Y., 197-205.

Smith and Waterman (1981) Ad. App. Math. 2:482.

Steinbuch and Audran (1969) Rev. Fr. Etud. Clin. Biol. 14:1054-1058.

Stewart and Yound (1984) Solid phase peptides synthesis, Pierce Chem. Company, Rockford.

Takeda et al. (1999) J. Immunol. 163:6269-6274.

Temin (1986) Retrovirus vectors for gene transfer. In Kucherlapati R., Ed. Gene Transfer.

Van de Waterbeemd, Advanced Computer-assisted techniques in drug discovery (Verlagsgesellschaft, Weinsheim, 1994).

Walker et al. (1982), EMBO J. 1:945-951.

1. An isolated polypeptide, named RH116, of amino acid sequence SEQ ID No. 2.

2. An isolated polypeptide, characterized in that it comprises a polypeptide chosen from:
a) a polypeptide of sequence SEQ ID No. 2;
b) a variant polypeptide of the polypeptide of amino acid sequences defined in a);
c) a polypeptide homologous to the polypeptide defined in a) or b) and comprising at least 80% identity, with said polypeptide of a);
d) a fragment of at least 15 consecutive amino acids of a polypeptide defined in a);
e) a biologically active fragment of a polypeptide defined in a).

3. The polypeptide as claimed in either one of claims 1 and 2, characterized in that it comprises at least one conserved domain belonging to the RNA helicase superfamily.

4. The polypeptide as claimed in claim 3, characterized in that said conserved domain is chosen from:
the G-GKT sequences corresponding to domain I of the RNA helicase superfamily,
the DEAD, DE-D and DEAH sequences corresponding to domain V of the RNA helicase superfamily.

5. A purified or isolated polynucleotide, characterized in that it encodes a polypeptide of claim 1.

6. The polynucleotide as claimed in claim 5, of sequence SEQ ID No. 1.

7. An isolated polynucleotide, characterized in that it comprises a polynucleotide chosen from:
a) SEQ ID No. 1;
b) the sequence of a fragment of at least 15 consecutive nucleotides of the sequence SEQ ID No. 1, with the exception of the registered nucleic acid sequences identified under the accession No. AC 007750 and No. AC0108176 in the GenBank databank, and the registered nucleic acid sequences identified under the accession Nos. AW589567, AW152541 and AW189584 in the EMBL databank;
c) a nucleic acid sequence exhibiting a percentage identity of at least 85%, after optimal alignment, with a sequence defined in a) or b);
d) the complementary sequence or the RNA sequence corresponding to a sequence as defined in a), b) or c).

8. The use of a polynucleotide as claimed in claim 7, as a primer for amplifying or polymerizing nucleic acid sequences.

9. The use, in vitro, of a polynucleotide as claimed in claim 7, as a probe for detecting nucleic acid sequences.

10. The use, in vitro, of a polynucleotide as claimed in claim 7, as a sense or antisense nucleic acid sequence for controlling the expression of the corresponding protein product.

11. The use of a polynucleotide as claimed in any one of claims 8, 9 and 10, characterized in that said polynucleotide is directly or indirectly labeled with a radioactive compound or a nonradioactive compound.

12. A recombinant cloning and/or expression vector, comprising a polynucleotide as claimed in one of claims 5 to 7 or encoding a polypeptide as claimed in any one of claims 1 to 4.

13. A recombinant antisense expression vector, comprising a polynucleotide as claimed in one of claims 5 to 7, characterized in that said polynucleotide is inserted in reverse orientation in said vector.

14. A host cell, characterized in that it is transformed with a vector as claimed in either of claims 12 and 13.

15. An animal, except a human, characterized in that it comprises a cell as claimed in claim 14.

16. A method for preparing a recombinant polypeptide, characterized in that a host cell as claimed in claim 14 is cultured under conditions which allow the expression and, optionally, the secretion of said recombinant polypeptide, and in that said recombinant polypeptide is recovered.

17. A recombinant polypeptide obtained using the method as claimed in claim 16.

18. A monoclonal or polyclonal antibody, and its fragments, characterized in that it selectively binds a polypeptide as claimed in one of claims 1 to 4 or 17.

19. A method for detecting and/or assaying a polypeptide as claimed in one of claims 1 to 4 or 17, in a biological sample, characterized in that it comprises the following steps:
a) bringing the biological sample into contact with an antibody as claimed in claim 18;
b) demonstrating the antigen-antibody complex formed.

20. A kit of reagents for carrying out a method as claimed in claim 19, in a biological sample, by immunoreaction, characterized in that it comprises the following elements:
a) a monoclonal or polyclonal antibody as claimed in claim 18;
b) where appropriate, the reagents for constituting the medium suitable for the immunoreaction;
c) the reagents for detecting the antigen-antibody complex produced during the immuno-reaction.

21. A method for detecting and/or assaying a polynucleotide as claimed in any one of claims 5 to 7, in a biological sample, characterized in that it comprises the following steps:
a) isolating the DNA from the biological sample to be tested, or obtaining a cDNA from the RNA of the biological sample;
b) specifically amplifying the DNA using primers as claimed in claim 8;
c) analyzing the amplification products.

22. A method for detecting and/or assaying a polynucleotide as claimed in any one of claims 5 to 7, in a biological sample, characterized in that it comprises the following steps:
a) bringing a polynucleotide as claimed in one of claims 5 to 7 into contact with a biological sample;
b) detecting and/or assaying the hybrid formed between said polynucleotide and the nucleic acid of the biological sample.

23. A DNA chip, characterized in that it contains a polynucleotide as claimed in one of claims 5 to 7.

24. A protein chip, characterized in that it contains a polypeptide as claimed in one of claims 1 to 4 or 17, or an antibody as claimed in claim 18.

25. A method for screening a compound which affects the level of cellular expression and/or the RNA helicase activity of a polypeptide as claimed in claims 1 to 4 or 17, and which comprises the steps of:
a) bringing a cell chosen from the, host cell of claim 14 and a eukaryotic cell, preferably a human cell, expressing or containing the polypeptide as claimed in claims 1 to 4 or 17, into contact with one or more potential compounds capable of penetrating or of being introduced into said cell;
b) detecting and/or measuring the level of cellular expression and/or the RNA helicase activity.

26. A method for screening a compound which affects the RNA helicase activity of a polypeptide as claimed in claims 1 to 4 or 17, and which comprises the steps of:
a) bringing said polypeptide into contact with one or more potential compound(s), in the presence of reagents required for implementing the RNA helicase activity;
b) detecting and/or measuring the RNA helicase activity.

27. The method as claimed in claims 25 and 26, characterized in that said screened compound decreases the level of expression and/or the RNA helicase activity of the polypeptide as claimed in claims 1 to 4 or 17.

28. A compound obtained using the method as claimed in claim 27, characterized in that it is chosen from:
a) a polynucleotide as claimed in any one of claims 5 to 7, used as an antisense nucleic acid sequence as claimed in claim 10;
b) an antisense expression vector as claimed in claim 13;
c) an antibody as claimed in claim 18;
d) an antagonist of the polypeptide as claimed in any one of claims 1 to 4 or 17;
e) muramyl peptides.

29. The compound as claimed in claim 28, characterized in that the muramyl peptide is. murabutide.

30. The method as claimed in claims 25 and 26, characterized in that said screened compound increases the level of expression and/or the RNA helicase activity of the polypeptide as claimed in claims 1 to 4 or 17.

31. A compound which is an agonist of the polypeptide as claimed in any one of claims 1 to 4 or 17, obtained using the method as claimed in claim 30.

32. A method for screening compounds which affect the functional activity of a polypeptide as claimed in claims 1 to 4 or 17, and which comprises the following steps of:
a) bringing said polypeptide into contact with one or more potential compound(s), in the presence of reagents required to carry out a reaction chosen from the nuclear and/or mitochondrial RNA splicing reaction, RNA editing reaction, rRNA processing reaction, translation initiation reaction, reaction of nuclear mRNA export to the cytoplasm and mRNA degradation reaction;
b) detecting and/or measuring said reaction.

33. A compound, characterized in that it is chosen from:
a) a polypeptide as claimed in one of claims 1 to 4 or 17;
b) a polynucleotide as claimed in one of claims 5 to 7;
c) a vector as claimed in claim 12 or 13;
d) a cell as claimed in claim 14;
e) an antibody as claimed in claim 18;
f) a compound as claimed in one of claims 28, 29 and 31;
as a medicinal product.

34. The compound as claimed in claim 33, as a medicinal product intended for the prevention and/or treatment of a pathology selected from the group composed of cancer, acute or chronic infectious diseases, hereditary genetic diseases, immune and auto-immune diseases, rheumatism, arthritis, artherosclerosis, osteoporosis and diabetes, and for the prevention of organ transplant rejection.

35. The compound as claimed in claim 34, characterized in that said infectious disease is selected from AIDS and hepatitis C.

36. The use of a compound as claimed in claim 33, for preparing a medicinal product intended for the treatment of viral pathologies.

37. The use as claimed in claim 36, characterized in that the viral pathology is acquired immunodeficiency syndrome (AIDS).

38. A pharmaceutical composition for the preventive and curative treatment of AIDS, characterized in that it contains a therapeutically effective amount of a compound as claimed in claim 33 and a pharmaceutically acceptable vehicle.

39. A product comprising at least one compound as claimed in claim 33 and at least one other antiviral agent, as a combination product for use simultaneously, separately or spread out over time in antiviral therapy, preferably anti-HIV therapy.

40. The use as claimed in claim 36, characterized in that the viral pathology is hepatitis B or C.

41. The use of a polynucleotide as claimed in any one of claims 5 to 7 and/or of a polypeptide as claimed in any one of claims 1 to 4 and 17 and/or of an agonist of a polypeptide as claimed in any one of claims 1 to 4 and 17, for preparing a medicinal product intended to cause or increase the immune response to a vaccine in a patient.
